SEQUENCE LISTING (26 sequences)

SEQ ID NO: 1 - DNA, 2211 bp, Homo sapiens
atggcggtacgcgcgttgaagctgctgaccacactgctggctgtcgtggccgctgcctcccaagccgaggtcgagtccgaggcaggatggggcatggtgacgcctgatctgctcttcgccgaggggaccgcagcctacgcgcgcggggactggcccggggtggtcctgagcatggaacgggcgctgcgctcccgggcagccctccgcgcccttcgcctgcgctgccgcacccagtgtgccgccgacttcccgtgggagctggaccccgactggtcccccagcccggcccaggcctcgggcgccggcgccctgcgcgacctgagcttcttcgggggccttctgcgtcgcgctgcctgcctgcgccgctgcctcgggccgccggccgcccactcgctcagcgaagagatggagctggagttccgcaagcggagcccctacaactacctgcaggtcgcctacttcaagatcaacaagttggagaaagctgttgctgcagcacacaccttcttcgtgggcaatcctgagcacatggaaatgcagcagaacctagactattaccaaaccatgtctggagtgaaggaggccgacttcaaggatcttgagactcaaccccatatgcaagaatttcgactgggagtgcgactctactcagaggaacagccacaggaagctgtgccccacctagaggcggcgctgcaagaatactttgtggcctatgaggagtgccgtgccctctgcgaagggccctatgactacgatggctacaactaccttgagtacaacgctgacctcttccaggccatcacagatcattacatccaggtcctcaactgtaagcagaactgtgtcacggagcttgcttcccacccaagtcgagagaagccctttgaagacttcctcccatcgcattataattatctgcagtttgcctactataacattgggaattatacacaagctggtgaatgtgccaagacctatcttctcttcttccccaatgacgaggtgatgaaccaaaatttggcctattatgcagctatgcttggagaagaacacaccagatccatcggcccccgtgagagtgccaaggagtaccgacagcgaagcctactggaaaaagaactgcttttcttcgcttatgatgtttttggaattccctttgtggatccggattcatggactccagaagaagtgattcccaagagattgcaagagaaacagaagtcagaacgggaaacagccgtacgcatctcccaggagattgggaaccttatgaaggaaatcgagacccttgtggaagagaagaccaaggagtcactggatgtgagcagactgacccgggaaggtggccccctgctgtatgaaggcatcagtctcaccatgaactccaaactcctgaatggttaccagcgggtggtgatggacggcgtaatctctgaccacgagtgtcaggagctgcagagactgaccaatgtggcagcaacctcaggagatggctaccggggtcagacctccccacatactcccaatgaaaagttctatggtgtcactgtcttcaaagccctcaagctggggcaagaaggcaaagttcctctgcagagtgcccacctgtactacaacgtgacggagaaagtgcggcgcatcatggagtcctacttccgcctggatacgcccctctacttttcctactctcatctggtgtgccgcactgccatcgaagaggtccaggcagagaggaaggatgatagtcatccagtccacgtggacaactgcatcctgaatgccgagaccctcgtgtgtgtcaaagagcccccagcctacaccttccgcgactacagcgccatcctttacctaaatggggacttcgatggcggaaacttttatttcactgaactggatgccaagaccgtgacggcagaggtgcagcctcagtgtggaagagccgtgggattctcttcaggcactgaaaacccacatggagtgaaggctgtcaccagggggcagcgctgtgccatcgccctgtggttcaccctggaccctcgacacagcgagcgggacagggtgcaggcagatgacctggtgaagatgctcttcagcccagaagagatggacctctcccaggagcagcccctggatgcccagcagggcccccccgaacctgcacaagagtctctctcaggcagtgaatcgaagcccaaggatgagctatga

SEQ ID NO: 2 - DNA, 2077 bp, Homo sapiens
atgcgggagcgcatctgggcgccgccgctgctgctgctgctgccgctgctactgccgccgccactgtggggcggccccccggacagcccacgccgggagctggagctggagcccgggcctctgcagcccttcgacctgctctacgccagcggcgcggccgcctactacagcggagactacgagcgagcggtgcgcgacttggaagcggcgctgcgcagccaccggcgcctgcgggaaatccgcacgcgctgtgcccgccactgcgcggcgcgccacccgctcccgcccccgccccccggcgagggccccggcgctgagctgccccttttccgctccttgttggggcgggcgcgctgttatcgcagctgtgagacccagcgcctcgggggccccgcatcccgccaccgcgtcagcgaggatgtgcgcagcgacttccagcgcagagtgccctacaactacctgcagcgggcctacatcaagcttaaccagctcgaaaaagcagtggaagcagctcacacatttttcgtggctaaccctgagcacatggaaatgcagcagaacattgagaattacagggcgacagctggtgttgaagcattgcagttggtagacagagaagccaagccacacatggagagttacaatgcaggagttaaacattatgaggctgatgactttgagatggctatcaggcatttcgaacaagccttaagagaatatttcgttgaagatacagaatgccggaccctatgtgaggggcctcagagatttgaagaatatgagtatttagggtataaggctggtctgtatgaagctattgcagatcactacatgcaggtgcttgtttgtcagcatgaatgtgtgagggaacttgccacccgccctggccgcctctctcccatcgagaattttcttcctctgcactatgattacctacagtttgcctactatcgagttggtgagtatgtgaaagccctggagtgtgccaaagcctatctgtcctagacaatgtggattactatgagagtctgctggatgatagcattgacccggcatccattgaggccagagaggatttaacaatgttcgtgaaacgtcataagctggagtctgagctgataaaatcagctgcagaaggtctggggttttcatacactgaaccgaattattggatcagatatggaggacgacaggatgagaatcgggtcccttcaggagtgaacgtagagggagcagaagttcatggattctcaatgggaaaaaagctatcacccaagatagatcgagacctaagagaaggtggtcctctactctatgagaacatcacattcgtctacaactcggagcagctgaacgggactcagcgggttctcctggataacgtcctgtcggaagaacagtgccgggagctccacagcgtggccagtggaatcatgcttgttggtgatggatacagaggaaaaacttcaccccatacacccaatgaaaagtttgaaggtgcaactgtcctgaaagcactcaaatctggttatgaaggtcgagtcccactgaagagcgctcgtctgttttatgacatcagcgaaaaggctcgaaggattgtagaatcttattttatgctgaactcaactctgtatttttcctatacacacatggtctgccgaacagccctgtctggtcagcaggatagaagaaatgacctcagtcatcccatccatgctgacaactgtttgttggatccagaggccaacgaatgctggaaggagcctcctgcttacacatttcgagactatagtgctctcctatatatgaatgatgactttgaaggaggagaattcatattcacagagatggatgctaagactgtgactgcctctataaaaccaaaatgtgggcgcatgatcagcttctcatctggaggagagaaccctcatggggtgaaggcagtcaccaagggaaagaggtgtgctgtggctctgtggttcaccttggacccactgctgatgaagtgattgcaattctggatcaagaacagcaagggaagcatgaactgaatatcaaccctaaagatgagctataa

SEQ ID NO: 3 - DNA, 2161 bp, Homo sapiens
atgctccggctcctccggccgctgctgctactgctgctgctgcctcccccggggtcccctgagccccccggcctgacccagctgtccccgggggcgcccccgcaggcccccgacttgctctacgctgacgggctgcgcgcctacgcggccggggcttgggcgccggccgtggcgctgctgcgggaggcgctgcggagccaggcggcgctgggccgggtgcggctggattgcggggcgagctgcgcggccgatccgggcgccgcgctccccgccgtgcttctcggggccccggagcccgactccgggccgggacccacgcaggggtcctgggagcgacagcttctccgtgcagcgctccgccgcgcagactgcctgacccagtgcgcagcacggaggctgggccccgggggcgcggcgcggcttcgcgtggggagcgcgctccgggacgccttccgccgtcgggagccctacaactacctgcagagggcctattaccagttgaagaagctggatctggcagctgcggcagcacacaccttctttgtagcaaaccccatgcacctgcagatgcgggaggacatggctaagtacagacgaatgtcgggagttcggccccagagcttccgggacctggagacgcccccacactgggcagcctatgacactggcctggagctactggggcgccaggaggcaggactggcactgcccaggctagaggaggctcttcaggggagcctggcccagatggagagctgccgtgctgactgtgaggggcctgaggagcagcagggggctgaagaagaggaggatggggctgcgagccaggggggcctctatgaggccattgcaggacactggattcaggtcctgcagtgccggcaacgctgtgtgggggaagcagccacacgccctggtcgcagcttccctgtcccagacttccttcccaaccagctgaggcggctacatgaggcccatgctcaggtgggcaatctgtcgtcctgctcttctacccggaggatgaggctgccaagagggctctgaaccagtaccaggcccagctgggagagccgagacctggcctcggacccagagaggacatccagcgcttcatcctccgatccctgggggagaagaggcagctctactatgccatggagcacctggggaccagcttcaaggatcctgacccctggacccctgcagctctcatccctgaggcacttagagaaaagctcagagaggatcaagagaagaggccttgggaccatgagcccgtgaagccaaagcccttgacctactggaaggatgtccttctcctggagggtgtgaccttgacccaggattccaggcagctgaatgggtcggagcgggcggtgttggatgggctgctcaccccagccgagtgtggggtgctgctgcagctggctaaggatgcagctggggctggagccaggtctggctatcgtggtcgccgctcccctcacaccccccatgaacgcttcgaggggctcacggtgcttaaggctgcgcagctggcccgggctgggacagtgggcagtcagggtgctaagctgcttctggaggtgagcgagcgggtgcggaccttgacccaggcctacttctccccggaacggcccctgcatctgtccttcacccacctggtgtgccgcagcgccatagaaggagagcaagagcagcgcatggacctgagtcacccagtgcacgcagacaactgcgtcctggaccctgacacgggagagtgctggcgggagcccccagcctacacctatcgggactacagcggactcctctacctcaacgatgacttccagggtggggacctgttcttcacggagcccaacgccctcactgtcacggctcgggtgcgtcctcgctgtgggcgccttgtggccttcagctccggtgtcgagaatccccatggggtgtgggccgtgactcggggacggcgctgtgccctggcactgtggcacacgagtggatagaagccaaagaactgctgcaggagtcacaggaggaggaggaagaggaagaggaagaaatgcccagcaaagacccttccccagagccccctagccgcaggcaccagagggtccaagacaagactggaagggcacctcgggttcgggaggagctgtga

SEQ ID NO: 4 - DNA, 2170 bp, Mus musculus
atggcggtgagcgaaaggaggctgctggcagcgatgctagcggtcgccgccgccgccgccttacgggttgcggcggagtctgagccgggatgggacgtggcagccccagacctgctttacgcagaggggaccgcggcctactcgcgcggggactggcccggggtggtcctgaacatggagcgggctctgcgctcgcgggcggccctgcgtgccctccgcctgcgctgccgcacacgctgtgccaccgaactgccgtgggcaccggacctggatctcggtccggaccccagcctgagccaggacccgggcgccgccgccctgcacgacctgcgcttcttcggagccgtgctgcgccgtgccgcctgcctacgccgctgcctcgggccgccctctgcccacttgctgagtgaagaactggacctggagttcaacaagcggagcccgtacaactacctgcaggtcgcctatttcaagataaacaagctggagaaagctgtggctgcggcacacaccttctttgtgggcaatcctgagcacatggagatgcggcagaacctcgactattaccaaaccatgtctggggtgaaggaggcagacttcagggatctcgaggccaagccccatatgcatgagtttcggctgggggtacgactctactcagaggagaagccacaggaagctgtgccccacctggaggcggcactgcaagagtactttgtggccgatgaggagtgccgtgccctctgcgaagggccctatgactacgacggctacaactacctagactacagcgctgacctcttccaggccatcacagatcattacgtccaggtcctcaactgtaagcagaactgtgtcacggagctggcttcccacccaagtagggaaaagccctttgaagacttcctcccttcacactataattacctacagtttgcctactacaacattgggaactatacacaagctattgaatgtgccaagacctacctcctcttcagaatctggcttattacacagccatgcttggagaagaagaggccagctccatcagccccagggagaatgccgaggaataccgacgtcgaagcctgttggagaaagaactgcttttcttcgcttatgacatttttggaattccctttgtggatccggactcatggactccagaagaggtgattcccaagagactgcaggagaaacagaagtcggaacgcgaaaccgcagtccgcatctcccaggagattggcaaccttatgaaggagatcgagaccctcgtggaggagaagaccaaggagtccttggatgtgagccgactgacgcgggaaggtggtcccctgctgtatgaaggcatcagtctcaccatgaattccaaagtcttgaatggctcccagcgggtggtgatggatggtgtgatctctgatgatgagtgccaggagctgcagagactgaccaatgcggcagcaacttcgggagatggctaccgaggtcagacctccccacacaccccaaatgaaaagttctatggtgttactgtcctcaaagctctcaagctcgggcaggaaggaaaagttcctctgcagagtgcccgcatgtactacaacgtgacagagaaggtgcggcgtgtcatggagtcctacttccgcctggacacgcccctctatttctcttattcccacctcgtgtgccgcactgcaatagaagagtcacaggctgagaggaaggacagtagccaccccgtccacgtggataactgcatcctgaatgccgaagccctcatgtgtatcaaggagcccccagcctacacgttccgggactacagcgccatcctttacctcaatggcgacttcgatggaggaaacttttacttcacagaactagatgccaagactgtgacggcagaggtgcagccccagtgtggaagggctgtgggattctcttctggcactgagaacccacatggagtgaaggctgtcaccagggggcagcgctgcgccctcggcacagtgagagagacagggtgcaggcagatgacctggtgaagatgctgttcagcccagaagaggtggacctcccccaggaacagcccctgcctggccagcagggctcgccagagcctggagaagagtctctgtcagacagagggtcgctgcacaaggatgagctatga

SEQ ID NO: 5 - DNA, 2062 bp, Mus musculus
atgcgggagagcacgtgggtgtcgctgctgttgctgctactgctgcccacaccgcagcggggcggcccccaggacggtcgccggtccccggagccagagcccgagcgcggcccgctgcagcccttcgacctgctctacgccagcggcgtggctgcctactacagcggcgactatgagcgagcggtgcgggacctggaagccgccctgagcagtcaccggcgcctacgggacatccgcacgcgctgcgcccgccactgcgcggcgcgccgtccgctcgcgccccccggcgccggccccggagccgagctgcccttcttccgcgcggtgctggagcgggcgcgctgctcccgcagctgccagagccagcgcctgggcggccccgcgtcccgccaccgcgtcagcgaggacgtgcgcagcgacttccaacgcagagttccctacaactacctgcagagggcctacatcaagctcaaccagctggaaaaggccatggaagcagctcacacgttcttcatggccaaccccgagcacatggaaatgcagcaggacctagaggactataaagccacagcacgagtggaagcccccctgctagaccgcgaagccaagccacacctggaaagctataatgctggagtcaaacattatgaagctgatgactttgaatccgcaatcaagtactttgaacaagctttaagagagtatttcaatgaagacatggagtgccgtgctctatgtgaggggcctcagagatttgaggaatatgagtacttagggtataaaggtggtctttacgaggcaatcgcagatcactatatgcaggttctggtctgtcagcatgaatgtgtgagggaactcgccacccgcccaggccgcctctcaccaatcgagaactttcttcccttgcattacgactacctccagtttgcctactacagagttggtgaatatgtgaaagccttggagtgcgccaaagcctacctgatgttccatccagagacttctatgagagtctgctggatgacagcactgatccagcatccattgaggcccgggaggatttaacagcatttgtcaaacgtcataaacttgaagctgaactgatcaagttagctgcagagggcctgggattttcatatgctgaaccgaattactggatcagctatggaggacggcaggatgagaaccgagtcccttcaggagtgaatatggatggggcagaagttcatggattgtcaatgggaaagaagtcaccgcccaagataggtcgagacctaagagaaggtggccctctgctctacgagaacatcaccttcgtctacaactctgagcagctgaacgggactcaacgggttcttctcgataatgtcctgtcgcaagagcagtgtcgggagctgcacagtgtggccaatggaatcatgctggttggtgatggataccgagggaaaacgtcaccccacacacccaatgagaagtttgaaggagcaactgttttaaaagcactcaaatttggttatgaaggccgggtgccactgaagagtgcaagactgttctatgacatcagcgagaaggctcggaagattgtggaatcctatttcatgctgaactcaaccctgtatttttcctatacacacatggtctgcagaacagccctgtctgggcagcaggatcgaagaaatgacctcagtcaccccattcatgcagacaactgcctgctggatccagaagccaatgaatgctggaaggagcctcccgcttacacctttcgggactacagtgctctcctgtatatgaatgacgactttgacggaggggaattcatcttcacagaaatggatgctaaaaccgtgactgcctctataaaaccaaagtgtggccgaatgatcagcttctcgtctggcggcgagaaccctcatggcgtgaaggcggtcaccagaggccagcgctgtgctgtggcactatggtttacactggaccctctttaccgagaactggagccatcttggatcaagaacagcgcgggaagcatggactgaatattaaccccaaagatgagctatag

SEQ ID NO: 6 - DNA, 2149 bp, Mus musculus
atgctccggctcctgcggttgttgcttttgctgctgctgcctcccccggggtcccccgagcctcccgagcccccgggcctggctcagttgtcaccggggtcgcctccccaggcccccgacttgctctacgcggacgggctgcgagcctactcggccggggcttgggcgcccgcagtggcgctgctgcgagaggcgctgcggagccgggcggcgctgggccgcgcgcgccaggagtgcggggcgagctgcgcggccgagccgggcgccgcgctcccctcccagctcctcggagccccacatcctgtctccgggcccggggtctgggaaccgctgctcctgcgcgccacgctccgccgcgccgagtgcctgacccagtgcgcggtgcggaggctgggcccggggggcgcggcgcgcctccgcgtggggagcgcgctgcgggatgccttccgccggcgggagccttacaactacctgcagagggcctactaccagttgaagaagctggacctggcagcttctgcagcacacaccttctttgtggcaaacccaacacacctgcagatgcgggaagacatggctaagtacagaaggatgtctgcaatccgaccccagagcttccgggacctggtgacgcccctatactgggcagcttatgacactggcctggagcttctggagcaacgggaggcagcactggctctaccccaactagaggaggccctgcaggggagcctggcccacatggagagctgccgtgctgcctgtgaggggcctgaggagcaccaaggggctgaagaagagggagaagggagccagggaggcctgtatgaagccattgcaggacactggatacgggttctgcagtgcaggcagcactgtgtggcagacacggccacccgtcctggtcgcagcttccctgtccaggacttcctcctcagccagctgagacggctgcacgaggcgtatgctcaggtggggaacatgtctcaggccatggatacccagaggatgaggctgccaaaaaggctctgaaccagtaccaaactcagttgggagagccaagacctgacctgggaccacgagaggacatccagcggttcatccttcgctccctcggagagaagagacagttatattacgccatggagcatctgggcactagcttcaaggacccggattcttggaccccggaggctctcatccctaaggcactgagagagaggctcagagaggatcaagagaagaagccctgggaccatcagcctccacagcagaagcccttggctcactggaaggatgcccttctgatggagggtgtgaccctcacgcaggacgcccagcagctgaatgggtcggagcgagctgtcttagatgggctgctaacttcagccgagtgtggggttctgctgcagctggccaaggatgcagcacaggctggagccaggtcaggctaccgtggccgccgctctcctcatagtcctcatgaacgctttgaggggctcacggtgctcaaggctgctcagctggcccgggcagggactgtgggcaggccaggcgctaagctgcttctggaggtaagtgagcgagtgcggactttgacccaggcctacttctccccggaacggccactgcatctttccttcacccacctggtgtgccgaagtgccatagaaggtgaacaagagcagcgtatggacctgagtcacccggttcatgcagacaactgtgtcctggaccccgacactggcgaatgctggcgggagcccccagcctacacctatcgagactatagtggactcctctacctcaatgatgacttcaagggaggcgacctgttcttcacgcagcccaacgccctcactgtcacggctcaggtccgtcctcggtgtgggcgccttgtggccttcagctctggtggtgagaatccccatggtgtatgggctgtgactcggggacggcgctgtgccctagcactgtggcacacgtgggcacctgagccaaagagctgctgcaggaggaagaggaggaagaagaggaggaagacattctcagcagagacccttccccagaacccccaagtcacaagcttcagcgagtccaggagaaagctgggaagccccgccgggtccgggtccgagaggaactgtga

SEQ ID NO: 7 - DNA, 2128 bp, Gallus gallus
atggcgctgctgctgccgctgctgccgctgctggtctgggcggcgggaccgccgggaccgctgggaccgccggagcactccgagccccctctgccggccgagcccccggacgcgctcttcgctgccggcgccgaggcgtacgcgcggggggactggcccgccgtggtgctgcagatggagcgggcgctgcgggcccgcgccgctatccgcgctcgatcggttcggtgccgcctgcgctgcgccaatgccaccgcggtggtaccgacggatggactcgagccgactctccacgatctgctgttctttcgggggctgctgcggcgcgccgcctgtctgcggggctgcgggcccacccagccctcccggtaccgcctgggcgaggagctggagcgagagttccgcaagcggagcccctacaactacctccaggtcgcctacttcaagatcaataaggtggcgaaggctgtggctgcagcccacaccttctttgtggccaaccccgagcacgtggagatgaagcagaacctggagtactaccagatgatggcgggcgtcagggagtccgacttcgctgacctggaggccaggccccacatgacagagttccgcctgggtgtcaggttctacagcgaggagcagccggctgctgctgtcctgcacctggagaaggcactgcaggaatactttgtggcagacaccgagtgccgtgctctctgtgaggggccctatgactatgaaggctacaactacctggagtacaacgcagacctgttccaggccatgacagatcactacatgcaggtgctgagctgcaagcagggctgcgtcacagagctcgcctcacagcctggccgggagaagcccctggaggatttcctgccctcacatttcaactacttgcagtttgcctactacaacaatgggaattatgaaaaagcgattgaatgtgccaaaacatacttgctcttcttcccaaatgatgaggtgatgaaccagaaggagaagacctggccaggcccattgagccccgaaaggaaatccaggcataccgccagcgaagcctcatggagaaggagctgctcttcttcagctacgacgtctttgggatcccatttgtggacccggacacgtggacgcccgaagaggtgataccaaagaggctgcgagagaaacaaaaggtggagcgggagacagcggcgcgcatctccgaggagatcggcaacctgatgaaggagatcgagacgctggtggaggagaaggcgaaggagtctgcagaaatgagcaagttcatccgagaaggtggccccttggtgtatgagggagccagcgtcaccatgaactccaaaagcctgaacgggtctcagcgcgtggtggtggacggagtcctctcagctgaggagtgccgggagctgcagagactcaccaacgcagctgcctcggcaggggatggctatcgtgggaagacatcgcctcacacccccagtgagaccttctacggtgtcaccgtcctgaaggccctcaagctgggccaggagggcaaggtccccctgcagagtgcccacctctactacaacgtgacggagaaggtgcggcacatgatggagtcctacttccgcctggaggtcccgctccatttctcctactcacacctggtgtgccgcacagccatcgatgagaagcaagaaggccggagtgacaacagccacgaggtgcatgtggacaactgcatcctcaacgcggaggccctggtgtgtgtgaaggaaccaccagcctacaccttccgggattacagtgccatcctctacctcaatggggactttgaagggggagctttctacttcacggagctggatgccaagacgcagactgcagaggtgcagccccagtgtggccgtgccgtgggcttctcctctggctcagagaacccccacggggtgaaggctgtgaccaaaggccaacgctgtgccattgccctctggttcaccctggacccacggcggacgatttggtgaagatgcttttcaggacggaagaggtggatttgctgcaggagacgagcgcggagcaggagccaacggctgccaccagcactgctggcttgcatgcagcagggaaggacgaactgtga

SEQ ID NO: 8 - DNA, 2035 bp, Gallus gallus
atggcgccggggagccggtcctggggggccgtgctgttgctagcggcgatgctgcccgcggcgtgcgggagctgcggagcggacggcggcccgctggagcccttcgacgcgctgtacgccagcggcgtggaagcgtactacggcggcgacttcgcgggcgcggcgcggtgcctggagcaagcgctgcgcagccggcgggagctgcgggcggagcggctgcggtgccggcggcgctgccgggggcaggtgcggctggcggcgctgggcgcggggcccgccggggagctgcccttcttcggggcgctgctgcggcgcgccggctgtctgcggagctgcgaggaaccgcgcctgggagccgcgtcccggcaccgcgccgccgaggaggtgcgctctgacttccagcgcagggtgccctacagctacctgcagcgcgcctacatccagctgaacaagctggaggaggcagcaaacgcagctcacaccttcttcatggcaaaccctgagcacatggagatacagcaggacattgagaactacaaaaccacagcagggaaagtcagcttgatcgacctggaggccaagccgcacatggaggattacagtgctggtgtcaggcactatgacaaggaggagtacgggttggccatcaccttcttggagcgtgcactggaagggtactatgctgaggatgaggattgccagatcatgtgcgaaggaccacagaggtttgaggaacacgaatacctggaatataaggctggcctctacgaagctattgcagatcactacatgcaagtcctggcctgcaaacatgactgcatccgagagctcgccacgcgctcgggccgcatctcacccatcgaaaacttccttcccctccactacgattacctgcagtttgcctattacagagtcggtgactacgtgaaagccctggagtgtgccaggtcctacctcctcttccacccggatgatgaagatgtcttggagaatgcagcaccgtggatccagccactatcaagcccagaaaggaagcaaaagcattgctgcgtcggcacaagctggagtcccacctcctgcgggtggctgcggttggcctggggttcacctacacggagccaaactactggaagaggtatggtgcccgccaagatgagcatagtgtcccatcgagtattagcagtgagccagaggatgggccccggctgtcgctaacaaagaaacccacaccaaaaccagatcgagagctgaaggaggggggtccacttctctacagcgacgtgaagtttgtctacaactcacagcagctgaacggcacccagcgagtgctgctggacaacgtcatctcagaggagcagtgtcgggagcttcacagagtggccagtgggatcatgttggctggagatggctataggggcaaaacctctccccatactccaaatgagagatttgaaggagccacggtcctcaaagccctcaagtatggctatgagggccgtgtcccactgaagagcgcccggctcttctacgacatcagcgagaaggcccgcaggatcgtcgagtcctacttcatgctcaactccacgctctacttctcctacactcacctggtgtgccgcaccgctctctctggccagcaggagaggaggaatgacctgagccaccccatccatgccgacaactgcctcctggaccccgaggccaacgagtgctggaaggagcctcctgcctacacctttcgggactacagtgccctcctctacatgaatgcagatttcgaaggtggcgaattcattttcactgagatggatgccaaaacagtgacggcatccatcaagcccaagtgtggacggatggtcagcttctcatccggtggtgagaacccacatggagtgaaggcagttaccaaagggcagcggtgcgctgtggctctatggtttaccttggacccactttacagagagctggagcgaatccaggcagatgaagtgatcgccgcagtgaaatgaacatcaacccaaaggacgagctatga

SEQ ID NO: 9 - protein, 736 aa, Homo sapiens [sequence omitted]

SEQ ID NO: 10 - protein, 708 aa, Homo sapiens
MRERIWAPPLLLLLPLLLPPPLWGGPPDSPRRELELEPGPLQPFDLLYASGAAAYYSGDYERAVRDLEAALRSHRRLREIRTRCARHCAARHPLPPPPPGEGPGAELPLFRSLLGRARCYRSCETQRLGGPASRHRVSEDVRSDFQRRVPYNYLQRAYIKLNQLEKAVEAAHTFFVANPEHMEMQQNIENYRATAGVEALQLVDREAKPHMESYNAGVKHYEADDFEMAIRHFEQALREYFVEDTECRTLCEGPQRFEEYEYLGYKAGLYEAIADHYMQVLVCQHECVRELATRPGRLSPIENFLPLHYDYLQFAYYRVGEYVKALECAKAYLLCHPDDEDVLDNVDYYESLLDDSIDPASIEAREDLTMFVKRHKLESELIKSAAEGLGFSYTEPNYWIRYGGRQDENRVPSGVNVEGAEVHGFSMGKKLSPKIDRDLREGGPLLYENITFVYNSEQLNGTQRVLLDNVLSEEQCRELHSVASGIMLVGDGYRGKTSPHTPNEKFEGATVLKALKSGYEGRVPLKSARLFYDISEKARRIVESYFMLNSTLYFSYTHMVCRTALSGQQDRRNDLSHPIHADNCLLDPEANECWKEPPAYTFRDYSALLYMNDDFEGGEFIFTEMDAKTVTASIKPKCGRMISFSSGGENPHGVKAVTKGKRCAVALWFTLDPLYRELERIQADEVIAILDQEQQGKHELNINPKDEL

SEQ ID NO: 11 - protein, 736 aa, Homo sapiens
MLRLLRPLLLLLLLPPPGSPEPPGLTQLSPGAPPQAPDLLYADGLRAYAAGAWAPAVALLREALRSQAALGRVRLDCGASCAADPGAALPAVLLGAPEPDSGPGPTQGSWERQLLRAALRRADCLTQCAARRLGPGGAARLRVGSALRDAFRRREPYNYLQRAYYQLKKLDLAAAAAHTFFVANPMHLQMREDMAKYRRMSGVRPQSFRDLETPPHWAAYDTGLELLGRQEAGLALPRLEEALQGSLAQMESCRADCEGPEEQQGAEEEEDGAASQGGLYEAIAGHWIQVLQCRQRCVGETATRPGRSFPVPDFLPNQLRRLHEAHAQVGNLSQAIENVLSVLLFYPEDEAAKRALNQYQAQLGEPRPGLGPREDIQRFILRSLGEKRQLYYAMEHLGTSFKDPDPWTPAALIPEALREKLREDQEKRPWDHEPVKPKPLTYWKDVLLLEGVTLTQDSRQLNGSERAVLDGLLTPAECGVLLQLAKDAAGAGARSGYRGRRSPHTPHERFEGLTVLKAAQLARAGTVGSQGAKLLLEVSERVRTLTQAYFSPERPLHLSFTHLVCRSAIEGEQEQRMDLSHPVHADNCVLDPDTGECWREPPAYTYRDYSGLLYLNDDFQGGDLFFTEPNALTVTARVRPRCGRLVAFSSGVENPHGVWAVTRGRRCALALWHTWAPEHREQEWIEAKELLQESQEEEEEEEEEMPSKDPSPEPPSRRHQRVQDKTGRAPRVREEL

SEQ ID NO: 12 - protein, 739 aa, Mus musculus
MAVSERRLLAAMLAVAAAAALRVAAESEPGWDVAAPDLLYAEGTAAYSRGDWPGVVLNMERALRSRAALRALRLRCRTRCATELPWAPDLDLGPDPSLSQDPGAAALHDLRFFGAVLRRAACLRRCLGPPSAHLLSEELDLEFNKRSPYNYLQVAYFKINKLEKAVAAAHTFFVGNPEHMEMRQNLDYYQTMSGVKEADFRDLEAKPHMHEFRLGVRLYSEEKPQEAVPHLEAALQEYFVADEECRALCEGPYDYDGYNYLDYSADLFQAITDHYVQVLNCKQNCVTELASHPSREKPFEDFLPSHYNYLQFAYYNIGNYTQAIECAKTYLLFFPNDEVMHQNLAYYTAMLGEEEASSISPRENAEEYRRRSLLEKELLFFAYDIFGIPFVDPDSWTPEEVIPKRLQEKQKSERETAVRISQEIGNLMKEIETLVEEKTKESLDVSRLTREGGPLLYEGISLTMNSKVLNGSQRVVMDGVISDDECQELQRLTNAAATSGDGYRGQTSPHTPNEKFYGVTVLKALKLGQEGKVPLQSARMYYNVTEKVRRVMESYFRLDTPLYFSYSHLVCRTAIEESQAERKDSSHPVHVDNCILNAEALMCIKEPPAYTFRDYSAILYLNGDFDGGNFYFTELDAKTVTAEVQPQCGRAVGFSSGTENPHGVKAVTRGQRCAIALWFTLDPRHSERDRVQADDLVKMLFSPEEVDLPQEQPLPGQQGSPEPGEESLSDRGSLHKDEL

SEQ ID NO: 13 - protein, 703 aa, Mus musculus
MRESTWVSLLLLLLLPTPQRGGPQDGRRSPEPEPERGPLQPFDLLYASGVAAYYSGDYERAVRDLEAALSSHRRLRDIRTRCARHCAARRPLAPPGAGPGAELPFFRAVLERARCSRSCQSQRLGGPASRHRVSEDVRSDFQRRVPYNYLQRAYIKLNQLEKAMEAAHTFFMANPEHMEMQQDLEDYKATARVEAPLLDREAKPHLESYNAGVKHYEADDFESAIKYFEQALREYFNEDMECRALCEGPQRFEEYEYLGYKGGLYEAIADHYMQVLVCQHECVRELATRPGRLSPIENFLPLHYDYLQFAYYRVGEYVKALECAKAYLMFHPDNEDVLDNVDFYESLLDDSTDPASIEAREDLTAFVKRHKLEAELIKLAAEGLGFSYAEPNYWISYGGRQDENRVPSGVNMDGAEVHGLSMGKKSPPKIGRDLREGGPLLYENITFVYNSEQLNGTQRVLLDNVLSQEQCRELHSVANGIMLVGDGYRGKTSPHTPNEKFEGATVLKALKFGYEGRVPLKSARLFYDISEKARKIVESYFMLNSTLYFSYTHMVCRTALSGQQDRRNDLSHPIHADNCLLDPEANECWKEPPAYTFRDYSALLYMNDDFDGGEFIFTEMDAKTVTASIKPKCGRMISFSSGGENPHGVKAVTRGQRCAVALWFTLDPLYRELERIQADEVIAILDQEQRGKHGLNINPKDEL

SEQ ID NO: 14 - protein, 732 aa, Mus musculus
MLRLLRLLLLLLLPPPGSPEPPEPPGLAQLSPGSPPQAPDLLYADGLRAYSAGAWAPAVALLREALRSRAALGRARQECGASCAAEPGAALPSQLLGAPHPVSGPGVWEPLLLRATLRRAECLTQCAVRRLGPGGAARLRVGSALRDAFRRREPYNYLQRAYYQLKKLDLAASAAHTFFVANPTHLQMREDMAKYRRMSAIRPQSFRDLVTPLYWAAYDTGLELLEQREAALALPQLEEALQGSLAHMESCRAACEGPEEHQGAEEEGEGSQGGLYEAIAGHWIRVLQCRQHCVADTATRPGRSFPVQDFLLSQLRRLHEAYAQVGNMSQAMENVLSVLLFYPEDEAAKKALNQYQTQLGEPRPDLGPREDIQRFILRSLGEKRQLYYAMEHLGTSFKDPDSWTPEALIPKALRERLREDQEKKPWDHQPPQQKPLAHWKDALLMEGVTLTQDAQQLNGSERAVLDGLLTSAECGVLLQLAKDAAQAGARSGYRGRRSPHSPHERFEGLTVLKAAQLARAGTVGRPGAKLLLEVSERVRTLTQAYFSPERPLHLSFTHLVCRSAIEGEQEQRMDLSHPVHADNCVLDPDTGECWREPPAYTYRDYSGLLYLNDDFKGGDLFFTQPNALTVTAQVRPRCGRLVAFSSGGENPHGVWAVTRGRRCALALWHTWAPEHSEQEWTEAKELLQEEEEEEEEEDILSRDPSPEPPSHKLQRVQEKAGKPRRVRVREEL

SEQ ID NO: 15 - protein, 725 aa, Gallus gallus
MALLLPLLPLLVWAAGPPGPLGPPEHSEPPLPAEPPDALFAAGAEAYARGDWPAVVLQMERALRARAAIRARSVRCRLRCANATAVVPTDGLEPTLHDLLFFRGLLRRAACLRGCGPTQPSRYRLGEELEREFRKRSPYNYLQVAYFKINKVAKAVAAAHTFFVANPEHVEMKQNLEYYQMMAGVRESDFADLEARPHMTEFRLGVRFYSEEQPAAAVLHLEKALQEYFVADTECRALCEGPYDYEGYNYLEYNADLFQAMTDHYMQVLSCKQGCVTELASQPGREKPLEDFLPSHFNYLQFAYYNNGNYEKAIECAKTYLLFFPNDEVMNQNLAYYTAVLGEDLARPIEPRKEIQAYRQRSLMEKELLFFSYDVFGIPFVDPDTWTPEEVIPKRLREKQKVERETAARISEEIGNLMKEIETLVEEKAKESAEMSKFIREGGPLVYEGASVTMNSKSLNGSQRVVVDGVLSAEECRELQRLTNAAASAGDGYRGKTSPHTPSETFYGVTVLKALKLGQEGKVPLQSAHLYYNVTEKVRHMMESYFRLEVPLHFSYSHLVCRTAIDEKQEGRSDNSHEVHVDNCILNAEALVCVKEPPAYTFRDYSAILYLNGDFEGGAFYFTELDAKTQTAEVQPQCGRAVGFSSGSENPHGVKAVTKGQRCAIALWFTLDPRHSERERVQADDLVKMLFRTEEVDLLQETSAEQEPTAATSTAGLHAAGKDEL

SEQ ID NO: 16 - protein, 694 aa, Gallus gallus
MAPGSRSWGAVLLLAAMLPAACGSCGADGGPLEPFDALYASGVEAYYGGDFAGAARCLEQALRSRRELRAERLRCRRRCRGQVRLAALGAGPAGELPFFGALLRRAGCLRSCEEPRLGAASRHRAAEEVRSDFQRRVPYSYLQRAYIQLNKLEEAANAAHTFFMANPEHMEIQQDIENYKTTAGKVSLIDLEAKPHMEDYSAGVRHYDKEEYGLAITFLERALEGYYAEDEDCQIMCEGPQRFEEHEYLEYKAGLYEAIADHYMQVLACKHDCIRELATRSGRISPIENFLPLHYDYLQFAYYRVGDYVKALECARSYLLFHPDDEDVLENAAYYEGLLEGTVDPATIKPRKEAKALLRRHKLESHLLRVAAVGLGFTYTEPNYWKRYGARQDEHSVPSSISSEPEDGPRLSLTKKPTPKPDRELKEGGPLLYSDVKFVYNSQQLNGTQRVLLDNVISEEQCRELHRVASGIMLAGDGYRGKTSPHTPNERFEGATVLKALKYGYEGRVPLKSARLFYDISEKARRIVESYFMLNSTLYFSYTHLVCRTALSGQQERRNDLSHPIHADNCLLDPEANECWKEPPAYTFRDYSALLYMNADFEGGEFIFTEMDAKTVTASIKPKCGRMVSFSSGGENPHGVKAVTKGQRCAVALWFTLDPLYRELERIQADEVIAMLDQEHVGRSEMNINPKDEL

SEQ ID NO: 17 - DNA, 2146 bp, Gallus gallus
  CDS (37)...(2146)
cgcctcccgctcctcctaccgctcggcggctcggccatggcctcgctcgtctgcctcctcctcctcctcctgctccccgccgccacgtcctcggtcgcgggccgcctggtgccctacgacctgctgtacgccgacggcgtgcgggcgtacttcgcccgggactgggggcgggcggccgagctgctgcagcgcgccctgtacagctacgcggggctgcgggcggcccgtctcgcctgccgggatgcctgcctccgagaggccgcctttctcggcgagccgccggccgcggggccctgggaagccgccctcttcgaccgggtgctgcagcgtgccgactgcctgcagcattgcctggggagccggctgggcgccgcgccctccgcgcaccgcgccagccttgttatccagcgggacttcgagcggagagagccctacaactacctccaggtggccttcttccagctgaagaagctggaccaggcagtgtctgctgctcacacgttctttgtcgccaatccccaacacctccagatgcgtgaggacatagagaaataccggcgtatgtcaggggtgaagtcagacaatttccgagacctggaggccgcaccgcactgggaagcatatgaggctggtatgcagcattacaatgcagatgagtacgtgcaggctgtggacagactggaggagtcactcagagaggcactgtcagcacttgaagagtgtcgtgctctgtgtgaagggccgctggaggatgaagaggaggaggatttgcagcctggcctgtatgaagccattgcagcccattatattcaggtgttgaagtgcaggcagcagtgcgtccttgaaattgccacaaagccagggtggatttctgccacagaagatttcataccgtctcatcttgatttactgcagtttgcatatgatcaagttgggaacccggcactagctgcggaatgtgctgcttcctacttgctcttctatcccacggatgagcccgcgcagaacttggacaagatacagctgtcacagcaagagagaatattcgtcattatgtacagagatctcttatggagaagaaattgatttactatgccacggagcatctaggaggaacttttaatgaccctcagccctggtctgtgttttttcccttgctcccgagagaggatcagaagaagcaaaagcaggaaactctggatatggaagagagagggaagagaggttctttgccttttgagggtatcacagtcaccatggattcccaacagatgaatggaacccagagggttgtgtttgacagagtcctgacagagtctgagtgtaaggacttgctccggttgacagaggcagcaagaggaacaggagatggctatcgagcgagacggtcccctcacaccccacatgagagatttgaagggttaaccgttctgaaggccatgcagctagcccagaatggggacgtggactggagagatgccaaattgcttctgcaggccagtgagaaatcacggaaaatcatagaatcctatttcactccaggaaagaaactgcatttctcattcacacaccttgtgtgccgcacagcagtagaagaagaacaggaaggtcgcatggatcttagtcatcctgtccatgctgataactgtctgttggatcctgaggggaaagagtgctggaaagagccacctgcctatgtctacagggactacagtggcattctctacctcaatgatgacttccgaggtgggagccttttcttcactgacatagacactgtgactgtcacggctaatgtgcatcctaagtgtgggagactggtagctttcagctctggcaaggagaatccccatggcgtgcgggcagtgagccaaggcaggcgctgtgccattgctctctggtacacgcactccgtagagcatgctgagcaggagcgggtgaaggcagaagagctgatggagcagagagctgtggaagagaaccagcctgatggagaagcagatcctccccagagcctcttgtttccggtggtgggaccaggcccacaagccagaagcataagtctgtctcggaaaggacgcagcaccccaagaagctgcgggccagagatgagttctaagtacatggggccctgggaaggactggc

SEQ ID NO: 18 - protein, 710 aa, Gallus gallus
MASLVCLLLLLLLPAATSSVAGRLVPYDLLYADGVRAYFARDWGRAAELLQRALYSYAGLRAARLACRDACLREAAFLGEPPAAGPWEAALFDRVLQRADCLQHCLGSRLGAAPSAHRASLVIQRDFERREPYNYLQVAFFQLKKLDQAVSAAHTFFVANPQHLQMREDIEKYRRMSGVKSDNFRDLEAAPHWEAYEAGMQHYNADEYVQAVDRLEESLREALSALEECRALCEGPLEDEEEEDLQPGLYEAIAAHYIQVLKCRQQCVLEIATKPGWISATEDFIPSHLDLLQFAYDQVGNPALAAECAASYLLFYPTDEPVLEKMKQYRAELGQDTAVTARENIRHYVQRSLMEKKLIYYATEHLGGTFNDPQPWSVFFPLLPREDQKKQKQETLDMEERGKRGSLPFEGITVTMDSQQMNGTQRVVFDRVLTESECKDLLRLTEAARGTGDGYRARRSPHTPHERFEGLTVLKAMQLAQNGDVDWRDAKLLLQASEKSRKIIESYFTPGKKLHFSFTHLVCRTAVEEEQEGRMDLSHPVHADNCLLDPEGKECWKEPPAYVYRDYSGILYLNDDFRGGSLFFTDIDTVTVTANVHPKCGRLVAFSSGKENPHGVRAVSQGRRCAIALWYTHSVEHAEQERVKAEELMEQRAVEENQPDGEEYHGADRSSRSSPEPLVSGGGTRPTSQKHKSVSERTQHPKKLRARDEF

SEQ ID NO: 19 - protein, 558 aa, Artificial Sequence
  Consensus sequence
LALLLLLLLLGGPLPPDLLYADGVAYAAGDWAVLEALRSRAALRAIRLRCRCAAEAGAELFFRALLRRACLRCRLGASRHRVSEELRDFRRPYNYLQRAYFKLNKLEKAVAAAHTFFVANPEHMEMQDLEYRMSGVKFRDLEAKPHMAYGVRYEEAVHLEALQEYFDEECRALCEGPFEEYYLEYQAGLYEAIADHYIQVLCKQCVELATRPGRPIEDFLPSHYYLQFAYYNVGNYAIECAKSYLLFHPDDEVNLYYALLGEAIPREDIFVRSLLEKELLFFAELGSFDPDWTPGVIPLREKKEEAIGKSIRREGGPLLYEGITLTNSQLNGSQRVVLDGVLSEEECRELRLAAAAAGDGYRGKTSPHTPNEKFEGLTVLKALKLGEGKVPLSARLFYDVSEKVRRIMESYFLESPLYFSYTHLVCRTAIEGQERDLSHPVHADNCLLDPEAECWKEPPAYTFRDYSALLYLNDDFDGGEFFFTELDAKTVTAVKPKCGRLVAFSSGENPHGVKAVTRGQRCAIALWFTLDPHSEERIQADELIMLEEDPVSEPKDEL

SEQ ID NO: 20 - protein, 9 aa, Artificial Sequence
  Synthetically generated peptide
GPXGSGSGK

SEQ ID NO: 21 - protein, 18 aa, Artificial Sequence
  Synthetically generated peptide
GPXGPXGPXGPXGSGSGK

SEQ ID NO: 22 - DNA, 23 bp, Artificial Sequence
  Synthetically generated oligonucleotide
caatgccaccgcggtggtaccga

SEQ ID NO: 23 - DNA, 23 bp, Artificial Sequence
  Synthetically generated oligonucleotide
aagcggagcccctacaactacct

SEQ ID NO: 24 - DNA, 23 bp, Artificial Sequence
  Synthetically generated oligonucleotide
gaagcgtactacggcggcgactt

SEQ ID NO: 25 - DNA, 23 bp, Artificial Sequence
  Synthetically generated oligonucleotide
gaggaggtgcgctctgacttcca

SEQ ID NO: 26 - protein, 5 aa, Artificial Sequence
  Exemplary motif
CXXXC

(19) United States
(12) Patent Application Publication
(10) Pub. No.: US 2005/0277167 A1
(43) Pub. Date: Dec. 15, 2005

(54) Prolyl 3-hydroxylases

(76) Inventors: Hans Peter Bachinger, Beaverton, OR (US); Janice Vranka, Portland, OR (US)

Correspondence Address: J. PETER FASSE;FISH & RICHARDSON P.C., 225 Franklin Street, Boston, MA, 02110, US

(21) Appl. No.: 11/078,189

(22) Filed: Mar. 11, 2005

Related U.S. Application Data
(60) Provisional application No. 60/552,409, filed on Mar. 11, 2004.

(51) Int. Cl. A01K 67/00; C07H 21/04; C12Q 1/26; C12P 21/06; C12N 9/06

(52) U.S. Cl. 435/25; 435/191; 435/69.1; 435/320.1; 435/325; 530/388.26; 800/8; 536/23.2

(57) ABSTRACT

The invention provides prolyl 3-hydroxlase nucleic acids and proteins, methods of using such nucleic acids and proteins, and transgenic animals.

### CROSS-REFERENCE TO RELATED APPLICATIONS

This application claims priority to U.S. Provisional Application No. 60/552,409, filed Mar. 11, 2004, the contents of which is incorporated herein by reference in its entirety.

### TECHNICAL FIELD

This invention relates to prolyl 3-hydroxlase nucleic acids and proteins, and methods of using such nucleic acids and proteins.

### BACKGROUND

Biosynthesis of collagen involves a number of unique post-translational modifications that are catalyzed by several specific enzymes. Hydroxylation of appropriate procollagen prolyl and lysyl residues to 4-hydroxyprolyl, 3-hydroxyprolyl, and hydroxylysyl residues are modifications that occur inside cells to ensure proper folding and assembly of procollagen. Specific endoplasmic reticulum (ER) resident proteins carry out these modifications. Among those proteins are prolyl 4-hydroxylase (P4H), prolyl 3-hydroxylase (P3H) and lysyl hydroxylase (LH), all of which belong to a group of 2-oxoglutarate dioxygenases that require Fe²⁺,2-oxoglutarate, O₂, and ascorbate for their activity.

### SUMMARY

The present invention is based, in part, on the identification and characterization of certain prolyl 3-hydroxylases (P3H). These enzymes hydroxylate proline residues in protein sequences to 3(S)hydroxyproline and are involved, for example, in collagen biosynthesis. Accordingly, the invention provides certain prolyl 3-hydroxlase nucleic acids and proteins, methods of using such nucleic acids and proteins, e.g., in screening methods and treatment of conditions and diseases, and transgenic animals.

In one aspect, the invention includes isolated nucleic acid molecules encoding a polypeptide that: (i) includes at least six and less than all of the amino acids of the sequence set forth in SEQ ID NO:9, 10, 11, 12, 13, 14, 15, 16 or 18; and (ii) displays P3H activity and substrate protein binding ability, wherein the substrate protein includes the sequence Gly-Pro-Hyp, e.g., Gly-Pro-Hyp-Gly-Ser-Gly-Ser-Gly-Lys (SEQ ID NO:20), or (Gly-Pro-Hyp)₄-Gly-Ser-Gly-Ser-Gly-Lys (SEQ ID NO:21). The isolated nucleic acid can further include a nucleic acid sequence that encodes a fusion partner, e.g., a hexa-histidine tag, a hemagglutinin tag, an immunoglobulin constant (Fc) region, a secretory sequence, or a detectable marker (e.g., β-galactosidase, invertase, green fluorescent protein, luciferase, chloramphenicol, acetyltransferase, beta-glucuronidase, exo-glucanase or glucoamylase).

In another aspect, the invention includes polypeptides encoded by isolated nucleic acid molecules described herein. For example, the present invention includes polypeptides that: (i) include at least six and less than all of the amino acids of the sequence set forth in SEQ ID NO:9, 10, 11, 12, 13, 14, 15, 16 or 18; and (ii) display P3H activity and substrate protein binding ability, wherein the substrate protein includes the sequence Gly-Pro-Hyp, e.g., Gly-Pro-Hyp-Gly-Ser-Gly-Ser-Gly-Lys (SEQ ID NO:20), or (Gly-Pro-Hyp)₄-Gly-Ser-Gly-Ser-Gly-Lys (SEQ ID NO:21). The polypeptide can further include a fusion partner, e.g., a hexa-histidine tag, a hemagglutinin tag, an immunoglobulin constant (Fc) region, a secretory sequence, or a detectable marker (e.g., β-galactosidase, invertase, green fluorescent protein, luciferase, chloramphenicol, acetyltransferase, beta-glucuronidase, exo-glucanase or glucoamylase).

In yet another aspect, the invention includes fusion proteins that include a polypeptide described herein. For example, the invention includes polypeptides that include (i) a first amino acid sequence comprising a prolyl 3 hydroxylase protein or fragment thereof; and (ii) a second amino acid sequence unrelated to the first amino acid sequence, wherein the fusion protein displays prolyl 3-hydroxylase activity and substrate protein binding ability, wherein the substrate protein includes the amino acid sequence Gly-Pro-Hyp, e.g., Gly-Pro-Hyp-Gly-Ser-Gly-Ser-Gly-Lys (SEQ ID NO:20), or (Gly-Pro-Hyp)₄-Gly-Ser-Gly-Ser-Gly-Lys (SEQ ID NO:21), or procollagen or fragment thereof. The first amino acid sequence can include SEQ ID NO:9, 10, 11, 12, 13, 14, 15, 16 or 18, or a fragment thereof. The substrate protein can include the amino acid sequence Gly-Pro-Hyp-Gly-Ser-Gly-Ser-Gly-Lys. The second amino acid sequence can be a hexa-histidine tag, a hemagglutinin tag, an immunoglobulin constant (Fc) region, a secretory sequence, or a detectable marker.

In still another aspect, the invention includes fusion proteins including: (i) a first amino acid sequence comprising a P3H protein or biologically active fragment thereof; and (ii) a second amino acid sequence unrelated to the first amino acid sequence, wherein the fusion protein displays P3H activity and substrate protein binding ability, wherein the substrate protein includes the amino acid sequence Gly-Pro-Hyp, e.g., Gly-Pro-Hyp-Gly-Ser-Gly-Ser-Gly-Lys (SEQ ID NO:20), or (Gly-Pro-Hyp)₄-Gly-Ser-Gly-Ser-Gly-Lys (SEQ ID NO:21). The first amino acid sequence can include SEQ ID NO:9, 10, 11, 12, 13, 14, 15, 16 or 18, or a fragment thereof.

In a further aspect, the invention includes methods for identifying a candidate compound that modulates P3H activity. The method includes: (a) providing a polypeptide that: (i) includes a P3H protein or a fragment thereof; and (ii) displays P3H activity and substrate protein binding ability; (b) contacting the polypeptide with the substrate protein in the presence of a test compound; and (c) comparing the level of P3H activity or binding activity of the polypeptide toward the substrate protein in the presence of the test compound with the level of P3H activity or binding activity in the absence of the test compound, wherein a different level of binding or hydroxylase activity in the presence of the test compound than in its absence indicates that the test compound is a candidate compound that modulates P3H activity. The polypeptide of (a) can include SEQ ID NO:9, 10, 11, 12, 13, 14, 15, 16 or 18, or a fragment thereof. The substrate can include the amino acid sequence Gly-Pro-Hyp, e.g., Gly-Pro-Hyp-Gly-Ser-Gly-Ser-Gly-Lys (SEQ ID NO:20), or (Gly-Pro-Hyp)₄-Gly-Ser-Gly-Ser-Gly-Lys (SEQ ID NO:21). The method can further include: (d) determining whether the candidate compound modulates in vivo the activity of a P3H polypeptide or collagen biosynthesis, wherein modulation indicates that the candidate compound is a P3H modulating agent. The test compound can be, e.g., a polypeptide, ribonucleic acid, small molecule, and/or deoxyribonucleic acid. In the method, (a) the polypeptide can be provided as a first fusion protein comprising the polypeptide fused to (i) a transcription activation domain of a transcription factor or (ii) a DNA-binding domain of a transcription factor; and (b) the substrate protein can be provided as a second fusion protein comprising a substrate protein fused to (i) a transcription activation domain of a transcription factor or (ii) a DNA-binding domain of a transcription factor, to interact with the first fusion protein; and binding of the polypeptide with the substrate protein can be detected as reconstitution of a transcription factor.

In one aspect, the invention includes methods for identifying a candidate compound that modulates P3H activity. The methods include (a) providing a polypeptide comprising a P3H protein or fragment thereof; (b) contacting the polypeptide or fragment thereof with a test compound; and (c) detecting binding between the polypeptide or fragment thereof with the test compound, wherein binding indicates that the test compound is a candidate compound that modulates P3H activity. The polypeptide can include the sequence set forth in SEQ ID NO:9, 10, 11, 12, 13, 14, 15, 16 or 18, or a biologically active fragment thereof. The methods can further include (d) determining whether the candidate compound modulates in vivo the activity of a P3H polypeptide or collagen biosynthesis, wherein modulation indicates that the candidate compound is a P3H modulating agent. The test compound can be, e.g., a polypeptide, ribonucleic acid, small molecule, and/or deoxyribonucleic acid. In the method, (a) the polypeptide can be provided as a first fusion protein comprising the polypeptide fused to (i) a transcription activation domain of a transcription factor or (ii) a DNA-binding domain of a transcription factor; and (b) the substrate protein can be provided as a second fusion protein comprising a substrate protein fused to (i) a transcription activation domain of a transcription factor or (ii) a DNA-binding domain of a transcription factor, to interact with the first fusion protein; and binding of the polypeptide with the substrate protein can be detected as reconstitution of a transcription factor.

In another aspect, the invention includes methods for identifying a candidate compound that modulates P3H activity. The methods include (a) contacting a nucleic acid encoding a P3H protein or fragment thereof with a test compound; and (b) detecting an interaction of the test compound with the nucleic acid, wherein an interaction indicates that the test compound is a candidate compound that modulates P3H activity. The method can further include (c) determining whether the candidate compound modulates in vivo the activity of a P3H polypeptide or collagen biosynthesis, wherein modulation indicates that the candidate compound is a P3H modulating agent. The nucleic acid can include a sequence that encodes SEQ ID NO:9, 10, 11, 12, 13, 14, 15, 16 or 18, or a fragment thereof. The test compound can be, e.g., a polypeptide, ribonucleic acid, small molecule, and/or deoxyribonucleic acid.

In yet another aspect, the invention includes pharmaceutical formulations including a candidate compound or P3H modulating agent, e.g., identified by a method(s) described herein, and optionally a pharmaceutically acceptable excipient.

In an additional aspect, the invention includes methods of treating a disorder or condition described herein in a patient, comprising administering a candidate compound, P3H modulating agent, or pharmaceutical formulation described herein to the patient.

In still another aspect, the invention includes methods of modulating (i.e., increasing or decreasing) collagen biosynthesis in an organism; The methods include administering to the organism a therapeutically effective amount of a pharmaceutical formulation described herein.

In a further aspect, the invention includes a method for modulating (i.e., increasing or decreasing) collagen biosynthesis in an organism. The method includes suppressing expression of P3H in the organism using an siRNA molecule(s). A target sequence can include the sequence (1) CAATGCCACCGCGGTGGTACCGA (SEQ ID NO:22); (2) AAGCGGAGCCCCTACAACTACCT (SEQ ID NO:23); (3) GAAGCGTACTACGGCGGCGACTT (SEQ ID NO:24); and/or (4) GAGGAGGTGCGCTCTGACTTCCA (SEQ ID NO:25).

In an additional aspect, the invention includes an siRNA molecule that is capable of targeting the sequence (1) CAATGCCACCGCGGTGGTACCGA (SEQ ID NO:22); (2) AAGCGGAGCCCCTACAACTACCT (SEQ ID NO:23); (3) GAAGCGTACTACGGCGGCGACTT (SEQ ID NO:24); and/or (4) GAGGAGGTGCGCTCTGACTTCCA (SEQ ID NO:25).

In one aspect, the invention includes antibodies capable of specifically binding to a P3H polypeptide.

In another aspect, the invention includes an isolated nucleic acid sequence including SEQ ID NO:7, SEQ ID NO:8, or SEQ ID NO:17, or a biologically active (e.g., substrate binding domain- or catalytic domain-encoding) fragment thereof.

In yet another aspect, the invention includes isolated nucleic acid sequences that encode a polypeptide including SEQ ID NO:15, SEQ ID NO:16, or SEQ ID NO:18, or a biologically active fragment (e.g., a substrate binding or catalytic domain) thereof.

In still another aspect, the invention includes isolated polypeptides including SEQ ID NO:15, SEQ ID NO:16, or SEQ ID NO:18, or a biologically active fragment (e.g., a substrate binding or catalytic domain) thereof.

In a further aspect, the invention includes transgenic non-human mammals (e.g., a mouse, rat, goat or cow), one or more of whose cells include a transgene encoding a P3H (e.g., a P3H1, P3H2 or P3H3), wherein the transgene is expressed in one or more (e.g., all) cells of the transgenic mammal such that the mammal exhibits a P3H1, P3H2- or P3H3-mediated disorder. The mammal can be a mosaic for cells comprising the transgene. The transgenic non-human mammal can have increased or decreased levels of expression of the P3H encoded by the transgene compared to a wild-type mammal. The transgene can comprise a disrupted P3H1, P3H2 or P3H3 sequence. The transgenic non-human mammal of can constitutively express the P3H transgene, and it may be expressed in a specific cell type.

In an additional aspect, the invention includes transgenic non-human mammals (e.g., a mouse, rat, goat, or cow) whose somatic and germ cells comprise a disrupted P3H gene (e.g., a P3H1, P3H2 or P3H3 gene), the disruption being sufficient to affect the expression or activity of P3H compared to a wild-type mammal, the disrupted gene being introduced into the transgenic mammal or an ancestor of the mammal at an embryonic stage, wherein the mammal, if homozygous for the disrupted gene exhibits a P3H (e.g., P3H1, P3H2- or P3H3)-mediated disorder. The somatic and germ cells can include a disrupted P3H1, P3H2 or P3H3 gene and the mammal can have decreased or no detectable P3H1, P3H2 or P3H3 expression or activity compared to a wild type mammal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIGS. 1A-1D are representations of amino acid sequences that illustrate the alignment of P3H family members. FIG. 1A is an alignment of human (H), mouse (M), and chicken (C) sequences of prolyl 3-hydroxylase family members created using Vector NTI® software. Protein family members are assigned “1”, “2”, or “3” based on sequence homologies across species. Accession numbers for the sequences are as follows: human P3H1 (leprecan): AF097432, mouse P3H1: AAH24047, chicken P3H1: AY463528, human P3H2 (MLAT4): NP_{—}060662, mouse P3H2: NP_{—}775555, chicken P3H2: AY463529, human P3H3 (GRCB): NP_{—}055077, and mouse P3H3: AY463530. Conserved residues of the active site domains of the prolyl 4-hydroxylases and lysyl hydroxylases are indicated with “*”, whereas other conserved residues are indicated with a “·”. A repeating CXXXC (SEQ ID NO:26) motif in the amino terminal half of the proteins is indicated with a “+”. FIGS. 1B-1D show an alignment that includes chicken P3H3 and a consensus sequence derived from all family members listed on the figure.

FIGS. 2A-2C are representations of sodium dodecyl sulfate polyacrylamide gel electrophoresis (SDS-PAGE) gels illustrating affinity purification of chicken P3H1 and coimmunoprecipitation of the proteins cyclophilin B (CYPB) and cartilage-associated protein (CRTAP). FIG. 2A is an SDS-PAGE gel showing protein bands found in an eluate following prolyl 3-hydroxylase purification using an antibody bound to agarose beads. FIG. 2B is a reducing SDS-PAGE illustrating two proteins that specifically eluted (FIG. 2B lanes 1-4), which were sequenced and determined to be cyclophilin B (CYPB at 21 kDa) and cartilage associated protein (CRTAP at 46 kDa apparent molecular weight) and chicken P3H1 that eluted in pH 2.5 glycine buffer (FIG. 2B lanes 5-8). FIG. 2C is a reducing SDS-PAGE gel illustrating that gelatin sepharose pooled fractions loaded onto the antibody column and eluted with pH 2.5 elution buffer contained all three proteins, P3H1, CYPB, and CRTAP (FIG. 2C, lanes 1 to 4), suggesting a specific association between these proteins.

FIGS. 3A-3C are graphs illustrating enzyme activity of chicken P3H1 as a function of enzyme concentration, time, and substrate concentration. FIG. 3A is a graph illustrating enzyme activity of P3H1 as a function of enzyme concentration. P3H1 enzyme activity, measured by the release of tritiated water (THO), was determined as a function of increasing amounts of enzyme and showed a linear relationship up to 200 μl of enzyme (approximately equal to a final enzyme concentration of 11.4 nM). FIG. 3B is a graph illustrating enzyme activity of P3H1 as a function of time. Enzyme activity was measured over a range of time points and appeared to reach its maximum at approximately 30 minutes. FIG. 3C is a graph illustrating enzyme activity of P3H1 as a function of substrate concentration. Enzyme activity was measured in relationship to varying substrate concentrations. FIG. 3C is the double reciprocal or Lineweaver Burk plot of 1/v vs. 1/[substrate] concentration. In this double-reciprocal plot the intercept on the x-axis is −1/Km. The Km was determined to be 179 μl of substrate per 2 ml of reaction volume.

FIGS. 4A-4I are representations of nucleic acid sequences of eight P3H family members. FIG. 4A: human P3H1 (leprecan). FIG. 4B: human P3H2 (MLAT4). FIG. 4C: human P3H3 (GRCB). FIG. 4D: mouse P3H1. FIG. 4E: mouse P3H2. FIG. 4F: mouse P3H3. FIG. 4G: chicken P3H1. FIG. 4H: chicken P3H2. FIG. 4I: chicken P3H3.

### DETAILED DESCRIPTION

The present invention is based, in part, on the isolation and characterization of proteins that exhibit P3H activity. P3H enzymes hydroxylate proline residues in protein sequences to 3(S)hydroxyproline and are involved in collagen biosynthesis. P3H nucleic acids and polypeptides are useful, for example, as targets for identifying compounds that modulate collagen biosynthesis.

I. Nucleic Acids and Proteins

In one aspect, the invention includes certain P3H nucleic acids. P3H nucleic acids include, for example, human P3H nucleic acid sequences, such as SEQ ID NO:1 (human P3H1), SEQ. ID. NO:2 (human P3H2), or SEQ ID NO: 3 (human P3H3); mouse P3H nucleic acid sequences, such as SEQ ID NO:4 (mouse P3H1), SEQ ID NO:5 (mouse P3H2) or SEQ ID NO:6 (mouse P3H3); and chicken P3H nucleic acid sequences, such as SEQ ID NO:7 (chicken P3H1), SEQ ID NO:8 (chicken P3H2) or SEQ ID NO:17 (chicken P3H3). Included within the present invention are fragments of P3H nucleic acids, e.g., a fragment of SEQ ID NOs:1, 2, 3, 4, 5, 6, 7, 8 or 17. Fragments of P3H nucleic acids may encode at least one useful fragment of a P3H polypeptide (e.g., a human, mouse, or chicken P3H polypeptide), such as a catalytic domain, binding domain, or other useful fragment. For example, a fragment of a P3H nucleic acid may encode a fragment of a P3H polypeptide having P3H activity (e.g., amino acids from about 409 to about 736 of SEQ ID NO:9, amino acids from about 414 to about 712 of SEQ ID NO:10, or amino acids from about 422 to about 736 of SEQ ID NO:11) or a fragment of the polypeptide having protein disulfide isomerase activity (e.g., amino acids from about 1 to about 408 of SEQ ID NO:9, amino acids from about 1 to about 414 of SEQ ID NO:10, or amino acids from about 1 to about 422 of SEQ ID NO:11), or any portion thereof.

P3H nucleic acids described herein include both RNA and DNA, including genomic DNA and synthetic (e.g., chemically synthesized) DNA. Nucleic acids can be double-stranded or single-stranded. Where single-stranded, the nucleic acid can be a sense strand or an antisense strand. Nucleic acids can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “isolated nucleic acid” means a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated P3H1 nucleic acid includes some or all of the 5′ non-coding (e.g., promoter) sequences that are immediately contiguous to the coding sequence. The term includes, for example, recombinant DNA that is incorporated into a vector, into an autonomously replicating plasmid or virus, or into the genomic DNA of a prokaryote or eukaryote, or which exists as a separate molecule (e.g., a genomic DNA fragment produced by PCR or restriction endonuclease treatment) independent of other sequences. It also includes a recombinant DNA that is part of a hybrid gene encoding an additional polypeptide sequence.

The term “purified” refers to a P3H nucleic acid (or P3H polypeptide) that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

In some embodiments, the invention includes nucleic acid sequences that are substantially identical to a P3H nucleic acid. A nucleic acid sequence that is “substantially identical” to a P3H nucleic acid is at least 75% identical (e.g., at least about 80%, 85% or 90% identical) to the P3H nucleic acid sequences represented by SEQ ID NO:1, 2, 3, 4, 5, 6, 7, or 8. For purposes of comparison of nucleic acids, the length of the reference nucleic acid sequence will be at least 50 nucleotides, but can be longer, e.g., at least 60 nucleotides, or more nucleotides.

To determine the percent identity of two amino acid or nucleic acid sequences, the sequences are aligned for optimal comparison purposes (i.e., gaps can be introduced as required in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences. The two sequences may be of the same length.

The comparison of sequences and determination of percent identity between two sequences can be accomplished using a mathematical algorithm. The percent identity between two amino acid sequences is determined using the Needleman and Wunsch ((1970) J. Mol. Biol. 48:444-453 ) algorithm which has been incorporated into the GAP program in the GCG software package, using a Blossum 62 scoring matrix with a gap penalty of 12, a gap extend penalty of 4, and a frameshift gap penalty of 5. Skilled practitioners will appreciate that the percent identity between two sequences can be determined using techniques similar to those described above, with or without allowing gaps. In calculating percent identity, only exact matches are counted.

In other embodiments, the invention includes variants and homologs of certain P3H nucleic acids, e.g., variants and homologs of P3H nucleic acid sequences represented by SEQ ID NO:1, 2, 3, 4, 5, 6, 7, or 8. The terms “variant” or “homolog” in relation to P3H nucleic acids include any substitution, variation, modification, replacement, deletion, or addition of one (or more) nucleotides from or to the sequence of a P3H nucleic acid. The resulting nucleotide sequence may encode a P3H polypeptide that is generally at least as biologically active as the referenced P3H polypeptides (e.g., as represented by SEQ ID NO:9, 10, 11, 12, 13, 14, 15, or 16). In particular, the term “homolog” covers homology with respect to structure and/or function, providing the resultant nucleotide sequence codes for or is capable of coding for a P3H polypeptide being at least as biologically active as P3H encoded by a sequence shown herein as SEQ ID NO:1, 2, 3, 4, 5, 6, 7, 8, or 17. With respect to sequence homology, there is at least 75% (e.g., 85%, 90%, 95%, 98%, or 100%) homology to the sequence shown as SEQ ID NOs:1, 2, 3, 4, 5, 6, 7, 8 or 17.

Also included within the scope of the present invention are alleles of a P3H gene. As used herein, an “allele” or “allelic sequence” is an alternative form of P3H. Alleles result from a mutation, i.e., a change in the nucleotide sequence, and generally produce altered mRNAs or polypeptides whose structure or function may or may not be altered. Any given gene can have none, one, or more than one allelic form. Common mutational changes that give rise to alleles are generally ascribed to deletions, additions or substitutions of amino acids. Each of these types of changes can occur alone, or in combination with the others, one or more times in a given sequence.

The invention also includes nucleic acids that hybridize, e.g., under stringent hybridization conditions (as defined herein) to all or a portion of the nucleotide sequences represented by SEQ ID NO:1, 2, 3, 4, 5, 6, 7, 8 or 17, or a complement thereof. The hybridizing portion of the hybridizing nucleic acids is typically at least 15 (e.g., 20, 30, or 50) nucleotides in length. The hybridizing portion of the hybridizing nucleic acid is at least about 75%, e.g., at least about 80%, 95%, 98% or 100%, identical to the sequence of a portion or all of a nucleic acid encoding an P3H polypeptide, or to its complement. Hybridizing nucleic acids of the type described herein can be used as a cloning probe, a primer (e.g., a PCR primer), or a diagnostic probe. Nucleic acids that hybridize to the nucleotide sequence represented by SEQ ID NO:1, 2, 3, 4, 5, 6, 7, 8 or 17, are considered “antisense oligonucleotides.”

High stringency conditions are hybridizing at 68° C. in 5×SSC/5× Denhardt's solution/1.0% SDS, or in 0.5 M NaHPO₄ (pH 7.2)/1 mM EDTA/7% SDS, or in 50% formamide/0.25 M NaHPO₄ (pH 7.2)/0.25 M NaCl/1 mM EDTA/7% SDS; and washing in 0.2×SSC/0.1% SDS at room temperature or at 42° C., or in 0.1×SSC/0.1% SDS at 68° C., or in 40 mM NaHPO₄ (pH 7.2)/1 mM EDTA/5% SDS at 50° C., or in 40 mM NaHPO₄ (pH 7.2) 1 mM EDTA/1% SDS at 50° C. Stringent conditions include washing in 3×SSC at 42° C. The parameters of salt concentration and temperature can be varied to achieve the optimal level of identity between the probe and the target nucleic acid. Additional guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

Also included in the invention are various engineered cells, e.g., transformed host cells, which contain a P3H nucleic acid described herein. A transformed cell is a cell into which (or into an ancestor of which) has been introduced, by means of recombinant DNA techniques, a nucleic acid encoding an P3H polypeptide. Both prokaryotic and eukaryotic cells are included, e.g., fungi, and bacteria, such as Escherichia coli, and the like.

Also included in the invention are genetic constructs (e.g., vectors and plasmids) that include a P3H nucleic acid described herein, operably linked to a transcription and/or translation sequence to enable expression, e.g., expression vectors. A selected nucleic acid, e.g., a DNA molecule encoding a P3H polypeptide, is “operably linked” to another nucleic acid molecule, e.g., a promoter, when it is positioned either adjacent to the other molecule or in the same or other location such that the other molecule can direct transcription and/or translation of the selected nucleic acid.

In another aspect, the invention includes certain P3H polypeptides. P3H polypeptides include, for example, human P3H polypeptides, such as those shown in SEQ ID NO:9 (human P3H1), SEQ ID NO:10 (human P3H2), and SEQ ID NO:11 (human P3H3)); mouse P3H polypeptides, such as those shown in SEQ ID NO:12 (mouse P3H1), SEQ ID NO:13 (mouse P3H2) and SEQ ID NO:14 (mouse P3H3); and chicken P3H polypeptides, such as those shown in SEQ ID NO:15 (chicken P3H1), SEQ ID NO:16 (chicken P3H2), and SEQ ID NO:18 (chicken P3H3). Included within the present invention are biologically active fragments of P3H polypeptides, e.g., fragments of SEQ ID NOs:9, 10, 11, 12, 13, 14, 15, 16 and 18. Fragments of P3H polypeptides may include at least one catalytic domain, binding domain, or other useful portion of a full-length P3H polypeptide. For example, useful fragments of P3H polypeptides include, but are not limited to, fragments of P3H polypeptides having P3H activity (e.g., amino acids from about 409 to about 736 of SEQ ID NO:9, amino acids from about 414 to about 712 of SEQ ID NO:10, or amino acids from about 422 to 736 of SEQ ID NO:11) and fragments P3H polypeptides having protein disulfide isomerase activity (e.g., amino acids from about 1 to about 408 of SEQ ID NO:9, amino acids from about 1 to about 414 of SEQ ID NO:10, or amino acids from about 1 to about 422 of SEQ ID NO:11), or any portions thereof.

The terms “protein” and “polypeptide” both refer to any chain of amino acids, regardless of length or post-translational modification (e.g., glycosylation or phosphorylation). Thus, the terms “P3H protein” and “P3H polypeptide” include full-length naturally occurring isolated P3H proteins, as well as recombinantly or synthetically produced polypeptides that correspond to the full-length naturally occurring proteins, or to a fragment of the full-length naturally occurring or synthetic polypeptide.

As discussed above, the term P3H polypeptide includes biologically active fragments of naturally occurring or synthetic P3H polypeptides. Fragments of a protein can be produced by any of a variety of methods known to those skilled in the art, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid that encodes the polypeptide. Expression of such mutagenized DNA can produce polypeptide fragments. Digestion with “end-nibbling” endonucleases can thus generate DNAs that encode an array of fragments. DNAs that encode fragments of a protein can also be generated, e.g., by random shearing, restriction digestion, chemical synthesis of oligonucleotides, amplification of DNA using the polymerase chain reaction, or a combination of the above-discussed methods. Fragments can also be chemically synthesized using techniques known in the art, e.g., conventional Merrifield solid phase FMOC or t-Boc chemistry. For example, peptides of the present invention can be arbitrarily divided into fragments of desired length with no overlap of the fragments, or divided into overlapping fragments of a desired length.

In certain embodiments, P3H polypeptides include a sequence substantially identical to all or a portion of a naturally occurring P3H polypeptide, e.g., a polypeptide that includes all or a portion of SEQ ID NO:9, 10, 11, 12, 13, 14, 15, 16, or 18. Polypeptides “substantially identical” to the P3H polypeptide sequences described herein have an amino acid sequence that is at least 65% identical to the amino acid sequence of the P3H polypeptide represented by SEQ ID NOs:9, 10, 11, 12, 13, 14, 15, 16 or 18 (measured as described herein). The polypeptides can also have a greater percentage identity, e.g., 75%, 85%, 90%, 95%, or even higher. For purposes of comparison, the length of the reference P3H polypeptide sequence can be at least 16 amino acids, e.g., at least 20 or 25 amino acids.

In the case of polypeptide sequences that are less than 100% identical to a reference sequence, the non-identical positions can be, but do not necessarily need to be, conservative substitutions for the reference sequence. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Where a particular polypeptide is said to have a specific percent identity to a reference polypeptide of a defined length, the percent identity is relative to the reference polypeptide. Thus, a polypeptide that is 50% identical to a reference polypeptide that is 100 amino acids long can be a 50 amino acid polypeptide that is completely identical to a 50 amino acid long portion of the reference polypeptide. It also might be a 100 amino acid long polypeptide that is 50% identical to the reference polypeptide over its entire length.

P3H polypeptides of the invention include, but are not limited to, recombinant polypeptides and natural polypeptides. Also included are nucleic acid sequences that encode forms of P3H polypeptides in which naturally occurring amino acid sequences are altered or deleted. Certain nucleic acids of the present invention may encode polypeptides that are soluble under normal physiological conditions. Also within the invention are nucleic acids encoding fusion proteins in which a portion of a P3H polypeptide is fused to an unrelated polypeptide (e.g., a marker polypeptide or a fusion partner) to create a fusion protein. For example, the polypeptide can be fused to a hexa-histidine tag to facilitate purification of bacterially expressed polypeptides or to a hemagglutinin tag to facilitate purification of polypeptides expressed in eukaryotic cells. The invention also includes, for example, isolated polypeptides (and the nucleic acids that encode these polypeptides) that include a first portion and a second portion; the first portion includes, e.g., a P3H polypeptide, and the second portion includes an immunoglobulin constant (Fc) region or a detectable marker.

The fusion partner can be, for example, a polypeptide that facilitates secretion, e.g., a secretory sequence. Such a fused polypeptide is typically referred to as a preprotein. The secretory sequence can be cleaved by the host cell to form the mature protein. Also within the invention are nucleic acids that encode a P3H polypeptide fused to a polypeptide sequence to produce an inactive preprotein. Preproteins can be converted into the active form of the protein by removal of the inactivating sequence.

II. Methods for Identifying Compounds Capable of Modulating P3H Activity

The invention provides screening methods for identifying compounds, e.g., small organic or inorganic molecules (M.W. less than 1,000 Da), oligopeptides, oligonucleotides, or carbohydrates, capable of modulating (i.e., reducing or increasing) P3H activity.

The invention also includes isolated compounds capable of modulating P3H activity. A purified or isolated compound is a composition that is at least 60% by weight the compound of interest. In general, the preparation is at least 75% (e.g., at least 90%, 95%, or even 99%) by weight the compound of interest. Purity can be measured by any appropriate standard method, e.g., column chromatography, polyacrylamide gel electrophoresis, or HPLC analysis.

Screening Methods

The invention provides methods for identifying compounds capable of modulating P3H activity. Although applicants to not intend to be bound by any particular theory as to the biological mechanism involved, such compounds are thought to modulate specifically (1) the function of a P3H polypeptide and/or (2) expression of the P3H gene.

In certain aspects, screening for such compounds is accomplished by (i) identifying from a group of test compounds those that bind to P3H, modulate an interaction between P3H and a substrate, and/or modulate (i.e., increase or decrease) transcription and/or translation of P3H; and, optionally, (ii) further testing such compounds for their ability to modulate the activity of P3H in vitro or in vivo. Test compounds that bind to P3H, modulate an interaction between P3H and a substrate, or modulate transcription and/or translation of P3H, are referred to herein as “candidate compounds.” Candidate compounds further tested and found to be capable of modulating in vivo the activity of a P3H polypeptide and/or collagen biosynthesis are considered “P3H modulating agents.” In the screening methods of the present invention, candidate compounds can be, but do not necessarily have to be, tested to determine whether they are P3H modulating agents. Assays of the present invention may be carried out in whole cell preparations and/or in ex vivo cell-free systems.

In one aspect, the invention includes a method for screening test compounds to identify compounds that bind to P3H polypeptides. Binding of a test compound to a P3H polypeptide can be detected, for example, in vitro by reversibly or irreversibly immobilizing the test compound(s) on a substrate, e.g., the surface of a well of a 96-well polystyrene microtitre plate. Methods for immobilizing polypeptides and other small molecules are well known in the art. For example, microtitre plates can be coated with a P3H polypeptide by adding the polypeptide in a solution (typically, at a concentration of 0.05 to 1 mg/ml in a volume of 1-100 μl) to each well, and incubating the plates at room temperature to 37° C. for a given amount of time, e.g., for 0.1 hour to 36 hours. Polypeptides not bound to the plate can be removed by shaking excess solution from the plate, and then washing the plate (once or repeatedly) with water or a buffer. Typically, the polypeptide is in water or a buffer. The plate can then be washed with a buffer that lacks the bound polypeptide. To block the free protein-binding sites on the plates, plates can be blocked with a protein that is unrelated to the bound polypeptide. For example, 300 μl of bovine serum albumin (BSA) at a concentration of 2 mg/ml in Tris-HCl can be used. Suitable substrates include those substrates that contain a defined cross-linking chemistry (e.g., plastic substrates, such as polystyrene, styrene, or polypropylene substrates from Corning Costar Corp. (Cambridge, Mass.), for example). If desired, a beaded particle, e.g., beaded agarose or beaded sepharose, can be used as the substrate. P3H can then be added to the coated plate and allowed to bind to the test compound (e.g., at 37° C. for 0.5-12 hours). The plate can then be rinsed as described above.

Binding of P3H to the test compound can be detected by any of a variety of art-known methods. For example, an antibody that specifically binds to a P3H polypeptide (i.e., an anti-P3H antibody, e.g., the monoclonal antibody described in Example 1, below) can be used in an immunoassay. If desired, the antibody can be labeled (e.g., fluorescently or with a radioisotope) and detected directly (see, e.g., West and McMahon, J. Cell Biol. 74:264, 1977). Alternatively, a second antibody can be used for detection (e.g., a labeled antibody that binds to the Fc portion of the anti-P3H antibody). In an alternative detection method, the P3H polypeptide is labeled (e.g., with a radioisotope, fluorophore, chromophore, or the like), and the label is detected. In still another method, a P3H polypeptide is produced as a fusion protein with a protein that can be detected optically, e.g., green fluorescent protein (which can be detected under UV light). In an alternative method, the polypeptide is produced as a fusion protein with an enzyme having a detectable enzymatic activity, such as horseradish peroxidase, alkaline phosphatase, β-galactosidase, or glucose oxidase. Genes encoding all of these enzymes have been cloned and are available for use by skilled practitioners. If desired, the fusion protein can include an antigen, which can be detected and measured with a polyclonal or monoclonal antibody using conventional methods. Suitable antigens include enzymes (e.g., horse radish peroxidase, alkaline phosphatase, and β-galactosidase) and non-enzymatic polypeptides (e.g., serum proteins, such as BSA and globulins, and milk proteins, such as caseins).

In various in vivo methods for identifying polypeptides that bind to a P3H polypeptide, the conventional two-hybrid assays of protein/protein interactions can be used (see e.g., Chien et al., Proc. Natl. Acad. Sci. USA, 88:9578, 1991; Fields et al., U.S. Pat. No. 5,283,173; Fields and Song, Nature, 340:245, 1989; Le Douarin et al., Nucleic Acids Research, 23:876, 1995; Vidal et al., Proc. Natl. Acad. Sci. USA, 93:10315-10320, 1996; and White, Proc. Natl. Acad. Sci. USA, 93:10001-10003, 1996). Generally, two-hybrid methods involve in vivo reconstitution of two separable domains of a transcription factor. One fusion protein contains the P3H polypeptide fused to either a transactivator domain or DNA binding domain of a transcription factor (e.g., of Gal4). The other fusion protein contains a test polypeptide fused to either the DNA binding domain or a transactivator domain of a transcription factor. Once brought together in a single cell (e.g., a yeast cell or mammalian cell), one of the fusion proteins contains the transactivator domain and the other fusion protein contains the DNA binding domain. Therefore, binding of the P3H polypeptide to the test polypeptide (i.e., candidate compound) reconstitutes the transcription factor. Reconstitution of the transcription factor can be detected by detecting expression of a gene (i.e., a reporter gene) that is operably linked to a DNA sequence that is bound by the DNA binding domain of the transcription factor. Kits for practicing various two-hybrid methods are commercially available (e.g., from Clontech; Palo Alto, Calif.).

In another aspect, the invention includes a method for screening test compounds to identify a compound that modulates a protein-protein interaction between P3H and a substrate polypeptide. A substrate polypeptide used in any method described herein is a naturally occurring or synthetic (or combination of both naturally occurring and synthetic) substrate polypeptide for prolyl 3-hydroxylase and/or an enzyme that demonstrates protein disulfide isomerase activity. For example, a substrate polypeptide can include at least one proline residue, e.g., a polypeptide that includes the amino acid sequence Gly-Pro-Hyp, e.g., a polypeptide that includes the sequence Gly-Pro-Hyp-Gly-Ser-Gly-Ser-Gly-Lys (SEQ ID NO:20), e.g., a polypeptide that includes the sequence (Gly-Pro-Hyp)₄-Gly-Ser-Gly-Ser-Gly-Lys (SEQ ID NO:21). Exemplary substrate polypeptide include any type of collagen, e.g., Type I collagen, Type II collagen, Type IV collagen, Type V collagen, Type X collagen, or fragments thereof. In the present method, a first compound is provided. The first compound is a P3H polypeptide or biologically active fragment thereof, or the first compound is a substrate polypeptide. A second compound is provided which is different from the first compound and which is labeled. The second compound is P3H polypeptide or biologically active fragment thereof, or the second compound is a substrate polypeptide. A test compound is provided. The first compound, second compound and test compound are contacted with each other. The amount of label bound to the first compound is then determined. A change in protein-protein interaction between the first compound and the second compound as assessed by label bound is indicative of the usefulness of the compound in modulating a protein-protein interaction between P3H and the substrate protein. In some embodiments, the change is assessed relative to the same reaction without addition of the test compound.

In certain embodiments, the first compound provided is attached to a solid support. Solid supports include, e.g., resins, e.g., agarose, beads, and multiwell plates. In certain embodiments, the method includes a washing step after the contacting step, so as to separate bound and unbound label.

In certain embodiments, a plurality of test compounds is contacted with the first compound and second compound. The different test compounds can be contacted with the other compounds in groups or separately. In certain embodiments, each of the test compounds is contacted with both the first compound and the second compound in separate wells. For example, the method can screen libraries of test compounds. Libraries of test compounds are discussed in further detail below. Libraries can include, e.g., natural products, organic chemicals, peptides, and/or modified peptides, including, e.g., D-amino acids, unconventional amino acids, and N-substituted amino acids. Typically, the libraries are in a form compatible with screening in multiwell plates, e.g., 96-well plates. The assay is particularly useful for automated execution in a multiwell format in which many of the steps are controlled by computer and carried out by robotic equipment. The libraries can also be used in other formats, e.g., synthetic chemical libraries affixed to a solid support and available for release into microdroplets.

In certain embodiments, the first compound is a P3H polypeptide or fragment thereof, and the second compound is a substrate polypeptide. In other embodiments, the first compound is substrate polypeptide, and the second compound is a P3H polypeptide or fragment thereof. The solid support to which the first compound is attached can be, e.g., sepharose beads, SPA beads (microspheres that incorporate a scintillant) or a multiwell plate. SPA beads can be used when the assay is performed without a washing step, e.g., in a scintillation proximity assay. Sepharose beads can be used when the assay is performed with a washing step. The second compound can be labeled with any label that will allow its detection, e.g., a radiolabel, a fluorescent agent, biotin, a peptide tag, or an enzyme fragment. The second compound can also be radiolabeled, e.g., with ¹²⁵I or ³H.

In certain embodiments, the enzymatic activity of an enzyme chemically conjugated to, or expressed as a fusion protein with, the first or second compound, is used to detect bound protein. A binding assay in which a standard immunological method is used to detect bound protein is also included. In certain other embodiments, the interaction of a P3H polypeptide and a substrate protein is detected by fluorescence resonance energy transfer (FRET) between a donor fluorophore covalently linked to P3H (e.g., a fluorescent group chemically conjugated to P3H, or a variant of green fluorescent protein (GFP) expressed as an P3H-GFP chimeric protein) and an acceptor fluorophore covalently linked to a substrate protein, where there is suitable overlap of the donor emission spectrum and the acceptor excitation spectrum to give efficient nonradiative energy transfer when the fluorophores are brought into close proximity through the protein-protein interaction of P3H and the substrate protein.

In other embodiments, the protein-protein interaction can be detected by reconstituting domains of an enzyme, e.g., beta-galactosidase (see Rossi et al, Proc. Natl. Acad. Sci. USA 94:8405-8410 (1997)).

In still other embodiments, the protein-protein interaction is assessed by fluorescence ratio imaging (Bacskai et al, Science 260:222-226 (1993)) of suitable chimeric constructs of P3H polypeptides and substrate proteins in cells, or by variants of the two-hybrid assay (Fearon et al, Proc Natl Acad Sci USA 89:7958-7962 (1992); Takacs et al, Proc Natl Acad Sci USA 90:10375-10379 (1993); Vidal et al, Proc Natl Acad Sci USA 93:10315-10320 (1996); Vidal et al, Proc Natl Acad Sci USA 93:10321-10326 (1996)) employing suitable constructs of P3H polypeptides and substrate proteins and tailored for a high throughput assay to detect compounds that inhibit the P3H/substrate interaction. These embodiments have the advantage that the cell permeability of compounds that act as modulators in the assay is assured.

In another aspect, the invention includes a method for high-throughput screening of candidate compounds to identify a compound that modulates the enzymatic activity of a P3H polypeptide. Substrate polypeptide (e.g., substrate protein comprising a proline residue, e.g., a polypeptide including the sequence Gly-Pro-Hyp, e.g., Gly-Pro-Hyp-Gly-Ser-Gly-Ser-Gly-Lys (SEQ ID NO:20) or (Gly-Pro-Hyp)₄-Gly-Ser-Gly-Ser-Gly-Lys (SEQ ID NO:21), e.g., procollagen) is provided. P3H polypeptides or fragments thereof having enzymatic activity are provided. A test compound is provided. The substrate polypeptide, the P3H or fragment thereof, and the test compound are contacted with each other in reaction media, e.g., buffers, under conditions that allow enzymatic activity of the P3H polypeptide. In certain embodiments, the P3H polypeptide is separated from the reaction media. After contacting, it is determined whether the P3H polypeptide displayed a change in enzymatic activity toward a substrate, e.g., as compared to a control.

In one embodiment, the enzymatic activity is prolyl 3 hydroxylation of a substrate polypeptide. In such an embodiment, a substrate polypeptide comprising a proline residue, e.g., including the sequence Gly-Pro-Hyp, e.g., including Gly-Pro-Hyp-Gly-Ser-Gly-Ser-Gly-Lys (SEQ ID NO:20) or (Gly-Pro-Hyp)₄-Gly-Ser-Gly-Ser-Gly-Lys (SEQ ID NO:21), can be coupled to activated sepharose beads. The substrate polypeptide/sepharose bead compound can then incubated with a P3H polypeptide and a test compound. Substrate polypeptide/sepharose beads can be removed from the reaction mixture, washed and hydrolyzed. Amino acid analysis can then be used for quantitation of 3(S)-hydroxyproline. If the substrate protein includes more or less 3-hydroxyproline, e.g., as compared to a control, the test compound is considered a candidate compound. Alternatively, substrate polypeptides can be synthesized with a biotin label to allow improved access for the enzymes to the substrate (no Sepharose bead). The polypeptide can be retrieved from the reaction mixture with streptavidin beads.

In other embodiments, the enzymatic activity is protein disulfide isomerase activity (see, e.g., Lambert et al., Biochem J. 213:235-43 (1983)). The assay is based on the kinetics of reactivation of scrambled RNAse. Additional studies were recently published (Woycechowsky et al., Biochemistry 42:5387-5394 (2003)), and it:was shown that the tripeptide CGC exhibits disulfide isomerase activity. Protein disulfide isomerase contains CXXC as its active site. Protein disulfide isomerase activity can be monitored by observing the absorbance change during a pH titration to determine the pKₐ values (Woycechowsky et al., 2003). Additionally, the isomerase activity can be determined by the scrambled RNAse method.

In certain embodiments, the substrate protein is labeled. The substrate protein can be labeled with any label that will allow its detection. In certain embodiments, substrate protein is radiolabeled, e.g., with tritium. In certain embodiments, determination of whether the substrate becomes hydroxylated in the presence of a P3H polypeptide is accomplished by determining the release of tritiated water in the reaction media, or the retention of radiolabel on the substrate protein. A change in release of tritiated water from the substrate protein by the P3H polypeptide in the presence of the test compound, as compared to release in its absence, is indicative of the usefulness of the compound in modulating P3H activity.

In still another aspect, the invention provides methods of identifying test compounds that modulate (e.g., increase or decrease) expression of a P3H polypeptide. The method includes contacting a P3H nucleic acid with a test compound and then measuring expression of the encoded P3H polypeptide. In a related aspect, the invention features a method of identifying compounds that modulate (e.g., increase or decrease) the expression of P3H polypeptides by measuring expression of a P3H polypeptide in the presence of the test compound or after the addition of the test compound in: (a) a cell line into which has been incorporated a recombinant construct including the P3H nucleic acid sequence (e.g., SEQ ID NO:1, 2, 3, 4, 5, 6, 7, 8, or 17) or fragment or an allelic variation thereof; or (b) a cell population or cell line that naturally selectively expresses P3H, and then measuring the activity of P3H and/or the expression thereof.

Since the P3H nucleic acids described herein have been identified, they can be cloned into various host cells (e.g., fungi, E. coli, or yeast) for carrying out such assays in whole cells. Similarly, conventional in vitro assays of P3H activity can be used with the P3H polypeptides of the invention.

In certain embodiments, an isolated nucleic acid molecule encoding a P3H is used to identify a compound that modulates (e.g., increases or decreases) the expression of P3H in vivo (e.g., in a P3H-producing cell). In such embodiments, cells that express P3H are cultured, exposed to a test compound (or a mixture of test compounds), and the level of P3H expression or activity is compared with the level of P3H expression or activity in cells that are otherwise identical but that have not been exposed to the test compound(s). Standard quantitative assays of gene expression and P3H activity, e.g., prolyl 3 hydroxylase activity, can be used.

Expression of a P3H polypeptide can be measured using art-known methods, for example, by Northern blot PCR analysis or RNAse protection analyses using a nucleic acid molecule of the invention as a probe. Other examples include enzyme-linked immunosorbent assay (ELISA), radioimmunoassay (RIA) and fluorescent activated cell sorting (FACS). The level of expression in the presence of the test molecule, compared with the level of expression in its absence, will indicate whether or not the test compound modulates the expression of P3H.

In still another aspect, the invention provides methods of screening test compounds utilizing cell systems that are sensitive to perturbation to one or several transcriptional/translational components. In one embodiment, the cell system is a modified P3H-expressing cell in which one or more of the transcriptional/translational components of the cell are present in an altered form or in a different amount compared with a corresponding wild-type P3H-expressing cell. This method involves examining a test compound for its ability to perturb transcription/translation in such a modified cell.

In certain embodiments, the method includes identifying candidate compounds that interfere with steps in P3H translational accuracy, such as maintaining a proper reading frame during translation and terminating translation at a stop codon. This method involves constructing cells in which a detectable reporter polypeptide can only be produced if the normal process of staying in one reading frame or of terminating translation at a stop codon has been disrupted. This method further involves contacting the cell with a test compound to examine whether it increases or decreases the production of the reporter polypeptide.

In other embodiments, the cell system is a cell-free extract and the method involves measuring transcription or translation in vitro. Conditions are selected so that transcription or translation of the reporter is increased or decreased by the addition of a transcription modifier or a translation modifier to the cell extract.

One method for identifying candidate compounds relies upon a transcription-responsive gene product. This method involves constructing a cell in which the production of a reporter molecule changes (i.e., increases or decreases) under conditions in which cell transcription of a P3H nucleic acid changes (i.e., increases or decreases). Specifically, the reporter molecule is encoded by a nucleic acid transcriptionally linked to a sequence constructed and arranged to cause a relative change in the production of the reporter molecule when transcription of a P3H nucleic acid changes. A gene sequence encoding the reporter may, for example, be fused to part or all of the gene encoding the transcription-responsive gene product and/or to part or all of the genetic elements that control the production of the gene product. Alternatively, the transcription-responsive gene product may stimulate transcription of the gene encoding the reporter, either directly or indirectly. The method further involves contacting the cell with a test compound, and determining whether the test compound increases or decreases the production of the reporter molecule in the cell.

Alternatively, the method for identifying candidate compounds can rely upon a translation-responsive gene product. This method involves constructing a cell in which cell translation of a P3H nucleic acid changes (i.e., increases or decreases). Specifically, the reporter molecule is encoded by a nucleic acid either translationally linked or transcriptionally linked to a sequence constructed and arranged to cause a relative increase or decrease in the production of the reporter molecule when transcription of a P3H nucleic acid changes. A gene sequence encoding the reporter may, for example, be fused to part or all of the gene encoding the translation-responsive gene product and/or to part or all of the genetic elements that control the production of the gene product. Alternatively, the translation-responsive gene product may stimulate translation of the gene encoding the reporter, either directly or indirectly. The method further involves contacting the cell with a test compound, and determining whether the test compound increases or decreases the production of the first reporter molecule in the cell.

For these and any methods described herein, a wide variety of reporters may be used, with typical reporters providing conveniently detectable signals (e.g., by spectroscopy). By way of example, a reporter gene may encode an enzyme that catalyses a reaction that alters light absorption properties.

Examples of reporter molecules include, but are not limited, to β-galactosidase, invertase, green fluorescent protein, luciferase, chloramphenicol, acetyltransferase, beta-glucuronidase, exo-glucanase and glucoamylase. Alternatively, radiolabeled or fluorescent tag-labeled nucleotides can be incorporated into nascent transcripts that are then identified when bound to oligonucleotide probes. For example, the production of the reporter molecule can be measured by the enzymatic activity of the reporter gene product, such as β-galactosidase.

The methods described above can be used for high throughput screening of numerous test compounds to identify candidate compounds. By high-throughput screening is meant that the method can be used to screen a large number of candidate compounds relatively easily and quickly. Skilled practitioners will appreciate that any of the methods described above can be automated. Having identified a test compound as a candidate compound, the candidate compound can be further tested to confirm whether it is a P3H modulating agent, i.e., to determine whether it can modulate P3H activity and/or collagen biosynthesis in vivo (e.g., using an animal, e.g., rodent, model system) if desired.

Test Compounds

As used herein, a “test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds; e.g., heteroorganic or organometallic compounds.

Test compounds can be screened individually or in parallel. An example of parallel screening is a high throughput drug screen of large libraries of chemicals. Such libraries of candidate compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds or sets of unique compounds. Alternatively, prior experimentation and anecdotal evidence can suggest a class or category of compounds of enhanced potential. A library can be designed and synthesized to cover such a class of chemicals.

The synthesis of combinatorial libraries is well known in the art and has been reviewed (see, e.g., E. M. Gordon et al., J. Med. Chem. (1994) 37:1385-1401; DeWitt, S. H.; Czamik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; Ellman, J. A. Acc. Chem. Res. (1996) 29:132; Gordon, E. M.; Gallop, M. A.; Patel, D. V. Acc. Chem. Res. (1996) 29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/10092, WO93/09668, WO91/07087, WO93/20242, WO94/08051).

Libraries of compounds can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis”, 2nd edition, Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy can result in a library of peptides, e.g., modulators, which can be used to prepare a library of test compounds of the invention. In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

Libraries of compounds can be screened to determine whether any members of the library have a desired activity, and, if so, to identify the active species. Methods of screening combinatorial libraries have been described (see, e.g., Gordon et al., J Med. Chem., supra). Soluble compound libraries can be screened by affinity chromatography with an appropriate receptor to isolate ligands for the receptor, followed by identification of the isolated ligands by conventional techniques (e.g., mass spectrometry, NMR, and the like). Immobilized compounds can be screened by contacting the compounds with a soluble receptor; preferably, the soluble receptor is conjugated to a label (e.g., fluorophores, colorimetric enzymes, radioisotopes, luminescent compounds, and the like) that can be detected to indicate ligand binding. Alternatively, immobilized compounds can be selectively released and allowed to diffuse through a membrane to interact with a receptor. Exemplary assays useful for screening libraries of test compounds are described above.

Medicinal Chemistry

Once a compound (or agent) of interest has been identified, standard principles of medicinal chemistry can be used to produce derivatives of the compound. Derivatives can be screened for improved pharmacological properties, for example, efficacy, pharmaco-kinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a candidate compound or agent and measure the effects of the modification on the efficacy of the compound or agent to thereby produce derivatives with increased potency. For an example, see Nagarajan et al. (1988) J. Antibiot. 41: 1430-8. Furthermore, if the biochemical target of the compound (or agent) is known or determined, the structure of the target and the compound can inform the design and optimization of derivatives. Molecular modeling software is commercially available (e.g., Molecular Simulations, Inc.) for this purpose.

III. Antibodies

The invention also features purified or isolated antibodies that bind, e.g., specifically bind, to a P3H polypeptide. An antibody “specifically binds” to a particular antigen, e.g., a P3H polypeptide, when it binds to that antigen, but recognizes and binds to a lesser extent (e.g., does not recognize and bind) to other molecules in a sample, e.g., a biological sample that includes a P3H polypeptide. An antibody exemplary of the type included in the present invention is described in Example 1, below. The antibody described in Example 1 is produced by a hybridoma.

P3H polypeptides (or antigenic fragments or analogs of such polypeptides) can be used to raise antibodies useful in the invention, and such polypeptides can be produced by recombinant or peptide synthetic techniques (see, e.g., Solid Phase Peptide Synthesis, supra; Ausubel et al., supra). In general, the polypeptides can be coupled to a carrier protein, such as KLH, as described in Ausubel et al., supra, mixed with an adjuvant, and injected into a host mammal. A ‘carrier’ is a substance that confers stability on, and/or aids or enhances the transport or immunogenicity of, an associated molecule. Antibodies can be purified, for example, by affinity chromatography methods in which the polypeptide antigen is immobilized on a resin.

In particular, various host animals can be immunized by injection of a polypeptide of interest. Examples of suitable host animals include rabbits, mice, guinea pigs, and rats. Various adjuvants can be used to increase the immunological response, depending on the host species, including but not limited to Freund's (complete and incomplete adjuvant), adjuvant mineral gels such as aluminum hydroxide, surface active substances such as lysolecithin, pluronic polyols, polyanions, peptides, oil emulsions, keyhole limpet hemocyanin, dinitrophenol, BCG (bacille Calmette-Guerin) and Corynebacterium parvum. Polyclonal antibodies are heterogeneous populations of antibody molecules derived from the sera of the immunized animals.

Antibodies of the invention include monoclonal antibodies, polyclonal antibodies, humanized or chimeric antibodies, single chain antibodies, Fab fragments, F(ab′)₂ fragments, and molecules produced using a Fab expression library.

Monoclonal antibodies (mAbs), which are homogeneous populations of antibodies to a particular antigen, can be prepared using P3H, and standard hybridoma technology (see, e.g., Kohler et al., Nature, 256:495, 1975; Kohler et al., Eur. J. Immunol., 6:511, 1976; Kohler et al., Eur. J. Immunol., 6:292, 1976; Hammerling et al., In Monoclonal Antibodies and T Cell Hybridomas, Elsevier, N.Y., 1981; Ausubel et al., supra).

In particular, monoclonal antibodies can be obtained by any technique that provides for the production of antibody molecules by continuous cell lines in culture, such as those described in Kohler et al., Nature, 256:495, 1975, and U.S. Pat. No. 4,376,110; the human B-cell hybridoma technique (Kosbor et al., Immunology Today, 4:72, 1983; Cole et al., Proc. Natl. Acad. Sci. USA, 80:2026, 1983); and the EBV-hybridoma technique (Cole et al., Monoclonal Antibodies and Cancer Therapy, Alan R. Liss, Inc., pp. 77-96, 1983). Such antibodies can be of any immunoglobulin class including IgG, IgM, IgE, IgA, IgD, and any subclass thereof. The hybridomas producing the mAbs of this invention can be cultivated in vitro or in vivo.

Once produced, polyclonal or monoclonal antibodies can be tested for recognition, e.g., specific recognition, of P3H in an immunoassay, such as a Western blot or immunoprecipitation analysis using standard techniques, e.g., as described in Ausubel et al., supra. Antibodies that specifically bind to a P3H polypeptide, or conservative variants thereof, are useful in the invention. For example, such antibodies can be used in an immunoassay to detect an P3H polypeptide in tissue samples and/or to reduce (e.g., eliminate) P3H activity in a patient.

Antibodies can be produced using fragments of P3H that appear likely to be antigenic, by criteria such as high frequency of charged residues. For example, such fragments can be generated by standard techniques of PCR, and can be cloned into a pGEX expression vector (Ausubel et al., supra). Fusion proteins can be expressed in E. coli and purified using a glutathione agarose affinity matrix as described in Ausubel, et al., supra.

If desired, several (e.g., two or three) fusions can be generated for each protein, and each fusion can be injected into at least two rabbits. Antisera can be raised by injections in a series, typically including at least three booster injections. Typically, the antisera is checked for its ability to immunoprecipitate a recombinant P3H polypeptide, or some unrelated control protein, e.g., glucocorticoid receptor, chloramphenicol acetyltransferase, or luciferase.

Techniques developed for the production of“chimeric antibodies” (Morrison et al., Proc. Natl. Acad. Sci., 81:6851, 1984; Neuberger et al., Nature, 312:604, 1984; Takeda et al., Nature, 314:452, 1984) can be used to splice the genes from a mouse antibody molecule of appropriate antigen specificity together with genes from a human antibody molecule of appropriate biological activity. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; and U.S. Pat. Nos. 4,946,778 and 4,704,692) can be adapted to produce single chain antibodies against a P3H polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments can include but are not limited to F(ab′)₂ fragments, which can be produced by pepsin digestion of the antibody molecule, and Fab fragments, which can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Polyclonal and monoclonal antibodies that specifically bind to an P3H polypeptide can be used, for example, to detect expression of P3H in various tissues of a patient. For example, a P3H polypeptide can be detected in conventional immunoassays of biological tissues or extracts. Examples of suitable assays include, without limitation, Western blotting, ELISAs, radioimmune assays, and the like.

IV. Pharmaceutical Compositions

The compounds and agents, nucleic acids, polypeptides, and antibodies, e.g., anti-P3H polypeptide antibodies (all of which can be referred to herein as “active compounds”), can be incorporated into pharmaceutical compositions. Such compositions typically include the compound, agent, nucleic acid molecule, polypeptides, and/or antibody, and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate and gelatin in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

For the compounds described herein, an effective amount, e.g. of a protein or polypeptide (i.e., an effective dosage), ranges from about 0.001 to 30 mg/kg body weight, e.g. about 0.01 to 25 mg/kg body weight, e.g. about 0.1 to 20 mg/kg body weight. The protein or polypeptide can be administered one time per week for between about 1 to 10 weeks, e.g. between 2 to 8 weeks, about 3 to 7 weeks, or for about 4, 5, or 6 weeks. The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of a protein, polypeptide, antibody, or other compound can include a single treatment or, preferably, can include a series of treatments.

For antibodies, a useful dosage is 0.1 mg/kg of body weight (generally 10 mg/kg to 20 mg/kg). Generally, partially human antibodies and fully human antibodies have a longer half-life within the human body than other antibodies. Accordingly, lower dosages and less frequent administration are possible. Modifications such as lipidation can be used to stabilize antibodies and to enhance uptake and tissue penetration. A method for lipidation of antibodies is described by Cruikshank et al. ((1997) J. Acquired Immune Deficiency Syndromes and Human Retrovirology 14:193).

If the compound is a small molecule, exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Nucleic acid molecules (e.g., P3H DNA) of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Diseases and Conditions and Treatments Therefor

A variety of diseases and conditions can be treating using the compositions and methods of the present invention. For example, the compositions and methods described herein can be used to treat diseases and conditions that have been linked to inappropriate or unregulated collagen production and/or maturation. These include pathological fibrosis or scarring (including endocardial sclerosis), idiopathic interstitial fibrosis, interstitial pulmonary fibrosis, perimuscular fibrosis, Symmers' fibrosis, pericentral fibrosis, hepatic fibrosis, kidney fibrosis, pulmonary fibrosis, fibrosis of bone marrow, myocardial fibrosis, hepatitis, dermatofibroma, binary cirrhosis, alcoholic cirrhosis, acute pulmonary fibrosis, idiopathic pulmonary fibrosis, acute respiratory distress syndrome, kidney fibrosis/glomerulonephritis, kidney fibrosis/diabetic nephropathy, scleroderma/systemic, scleroderma/local, keloids, hypertrophic scars, severe joint adhesions/arthritis, myelofibrosis, corneal scarring, cystic fibrosis, muscular dystrophy (duchenne's), cardiac fibrosis, muscular fibrosis/retinal separation, esophageal stricture and payronles disease. Further fibrotic disorders may be induced or initiated by surgery, including scar revision/plastic surgeries, glaucoma, cataract fibrosis, corneal scarring, joint adhesions, graft vs. host disease, tendon surgery, nerve entrapment, dupuytren's contracture, OB/GYN adhesions/fibrosis, pelvic adhesions, peridural fibrosis, restenosis. Other conditions involving collagen production include ankylosing spondylitis, fibromuscular dysplasia, dermal scarring, and wounds.

Further, skilled practitioners will appreciate that increasing P3H activity can be useful in treating degenerative diseases of connective tissues and developing connective tissues during growth of a patient (e.g., to treat chondrodysplasia). Exemplary of degenerative diseases are osteoporosis, osteoarthitis, degeneration of subcuteneous tissues in aging, and degeneration of teeth and sclera.

One strategy for treating patients having conditions that involve inappropriate collagen production is to modulate the production of collagen in the patient. The goal is to normalize collagen production in the patient, i.e., to increase production where production is too low and to decrease production where production is too high. Modulation of collagen synthesis falls into two basic categories: inhibiting (i.e., reducing, e.g., eliminating) collagen synthesis and increasing (i.e., supplementing or providing) collagen synthesis where there is insufficient or no synthesis. Whether collagen synthesis should be inhibited or increased depends upon the intended application. The present invention provides methods for modulating P3H activity, and therefore collagen production, in a patient using the active compounds (e.g., candidate compounds and/or P3H modulating agents) described herein.

In certain aspects, the invention provides methods for inhibiting collagen biosynthesis, e.g., in a patient. Agents that inhibit collagen biosynthesis can be used, e.g., as treatments for scleroderma and related disorders, hepatic fibrosis, kidney fibrosis, pulmonary fibrosis, fibrosis of bone marrow, and keloid (skin fibrosis). In certain other aspects, the invention provides methods for increasing collagen synthesis. Compounds that increase synthesis can be used, e.g., as treatments to promote wound healing.

The term “patient” is used throughout the specification to describe an animal, human or non-human, rodent or non-rodent, to whom treatment according to the methods of the present invention is provided. Veterinary and non-veterinary applications are contemplated. The term includes, but is not limited, to birds (e.g., chickens), reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs.

Inhibition of P3H Activity

An antisense nucleic acid effective to inhibit expression of an endogenous P3H gene can be utilized. As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA.

Antisense molecules are designed so as to interfere with transcription or translation of a target gene upon hybridization with the target gene or transcript. The antisense nucleic acid can include a nucleotide sequence complementary to an entire P3H RNA or only a portion of the RNA. On one hand, the antisense nucleic acid needs to be long enough to hybridize effectively with P3H RNA. Therefore, the minimum length is approximately 12 to 25 nucleotides. On the other hand, as length increases beyond about 150 nucleotides, effectiveness at inhibiting translation may increase only marginally, while difficulty in introducing the antisense nucleic acid into target cells may increase significantly. Accordingly, an appropriate length for the antisense nucleic acid may be from about 15 to about 150 nucleotides, e.g., 20, 25, 30, 35, 40, 45, 50, 60, 70, or 80 nucleotides. The antisense nucleic acid can be complementary to a coding region of P3H mRNA or a 5′ or 3′ non-coding region of a P3H mRNA, or both. One approach is to design the antisense nucleic acid to be complementary to a region on both sides of the translation start site of the P3H mRNA.

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a P3H nucleic acid can be prepared, followed by testing for inhibition of P3H expression. Optionally, gaps of 5-10 nucleotides can be left between the oligonucleotides to reduce the number of oligonucleotides synthesized and tested.

The antisense nucleic acid can be chemically synthesized, e.g., using a commercial nucleic acid synthesizer according to the vendor's instructions. Alternatively, the antisense nucleic acids can be produced using recombinant DNA techniques. An antisense nucleic acid can incorporate only naturally occurring nucleotides. Alternatively, it can incorporate variously modified nucleotides or nucleotide analogs to increase its in vivo half-life or to increase the stability of the duplex formed between the antisense molecule and its target RNA. Examples of nucleotide analogs include phosphorothioate derivatives and acridine-substituted nucleotides. Given the description of the targets and sequences, the design and production of suitable antisense molecules is within ordinary skill in the art. For guidance concerning antisense nucleic acids, see, e.g., Goodchild, “Inhibition of Gene Expression by Oligonucleotides,” in Topics in Molecular and Structural Biology, Vol. 12. Oligodeoxynucleotides (Cohen, ed.), MacMillan Press, London, pp. 53-77.

Delivery of antisense oligonucleotides can be accomplished by any method known to those of skill in the art. For example, delivery of antisense oligonucleotides for cell culture and/or ex vivo work can be performed by standard methods such as the liposome method or simply by addition of membrane-permeable oligonucleotides. To resist nuclease degradation, chemical modifications such as phosphorothionate backbones can be incorporated into the molecule.

Delivery of antisense oligonucleotides for in vivo applications can be accomplished, for example, via local injection of the antisense oligonucleotides at a selected site. This method has previously been demonstrated for psoriasis growth inhibition and for cytomegalovirus inhibition. See, for example, Wraight et al., (2001). Pharmacol Ther. April; 90(1):89-104.; Anderson, et al., (1996) Antimicrob Agents Chemother 40: 2004-2011; and Crooke et al., J Pharmacol Exp Ther 277: 923-937.

Similarly, RNA interference (RNAi) techniques can be used to inhibit P3H, in addition or as an alternative to, the use of antisense techniques. For example, small interfering RNA (siRNA) duplexes directed against P3H nucleic acids could be synthesized and used to prevent expression of the encoded protein(s). Exemplary P3H sequences against which siRNA sequences can be directed include, but are not limited to:

(1) CAATGCCACCGCGGTGGTACCGA; (SEQ ID NO:22)
(2) AAGCGGAGCCCCTACAACTACCT; (SEQ ID NO:23)
(3) GAAGCGTACTACGGCGGCGACTT; (SEQ ID NO:24)
and
(4) GAGGAGGTGCGCTCTGACTTCCA. (SEQ ID NO:25)

As another example, P3H activity can be inhibited using a P3H polypeptide binding molecule such as an antibody, e.g., an anti-P3H polypeptide antibody, or a P3H polypeptide-binding fragment thereof. The anti-P3H polypeptide antibody can be polyclonal or monoclonal. An exemplary monoclonal anti-P3H polypeptide antibody is described in Example 1, below. Skilled practitioners will appreciate that such an antibody could be administered to patients, e.g., as-is or, preferably, modified (e.g., as discussed below) for administration to animals, e.g., humans.

Alternatively or in addition, the antibody can be produced recombinantly, e.g., produced by phage display or by combinatorial methods as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

As used herein, the term “antibody” refers to a protein comprising at least one, e.g., two, heavy (H) chain variable regions (abbreviated herein as VH), and at least one, e.g., two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

An anti-P3H polypeptide antibody can further include a heavy and light chain constant region, to thereby form a heavy and light immunoglobulin chain, respectively. The antibody can be a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2, and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

A “P3H polypeptide-binding fragment” of an antibody refers to one or more fragments of a full-length antibody that retain the ability to specifically bind to P3H polypeptide or a portion thereof. “Specifically binds” means that an antibody or ligand binds to a particular target and not to other unrelated substances, except in an easily reversible or “background” type interaction. Examples of P3H polypeptide binding fragments of an anti-P3H polypeptide antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “P3H polypeptide-binding fragment” of an antibody. These antibody fragments can be obtained using conventional techniques known to those with skill in the art.

Anti-P3H polypeptide antibodies can be fully human antibodies (e.g., an antibody made in a mouse which has been genetically engineered to produce an antibody from a human immunoglobulin sequence), or a non-human antibody, e.g., a rodent (mouse or rat), goat, primate (e.g., monkey), camel, donkey, porcine, or fowl antibody.

An anti-P3H polypeptide antibody can be one in which the variable region, or a portion thereof, e.g., the CDRs, are generated in a non-human organism, e.g., a rat or mouse. The anti-P3H polypeptide antibody can also be, for example, chimeric, CDR-grafted, or humanized antibodies. The anti-P3H polypeptide antibody can also be generated in a non-human organism, e.g., a rat or mouse, and then modified, e.g., in the variable framework or constant region, to decrease antigenicity in a human.

Another approach to inhibiting P3H activity is the administration of a P3H antagonist that binds to (i.e., blocks) P3H polypeptides and prevents them from interacting with a target protein (e.g., procollagen). Such P3H polypeptide antagonists can be identified using a screening method described herein. Alternatively, the P3H polypeptide antagonist can be an anti-P3H polypeptide antibody, or fragment thereof, as described above.

Increasing P3H Activity

New or supplemental P3H activity can be provided in vivo by direct administration of a naturally occurring and/or recombinant P3H polypeptide to a patient. P3H polypeptides that can be used to supplement P3H activity, e.g., in humans, are described herein, e.g., SEQ ID NO:9, 10, or 11, or fragments thereof. Other exemplary P3H polypeptides are described in Example 1, below. Such polypeptides can be used in modified or unmodified form. Examples of typical modifications are derivation of amino acid side chains, glycosylation, conservative amino acid substitutions, and chemical conjugation or fusion to other non-P3H polypeptide moieties.

Alternatively or in addition, a P3H polypeptide can be generated directly within an organism, e.g., a human, by expressing within the cells of the organism a nucleic acid construct containing a nucleotide sequence encoding a P3H polypeptide. Any appropriate expression vector suitable for transfecting the cells of the organism of interest can be used for such purposes. The nucleic acid construct can be derived from a non-replicating linear or circular DNA or RNA vector, or from an autonomously replicating plasmid or viral vector. Methods for constructing suitable expression vectors are known in the art, and useful materials are commercially available.

Another approach to increasing P3H activity is the administration of a compound identified as increasing P3H activity using a screen described herein.

VI. Transoenic Animals

The present invention also features transgenic animals that express P3H polypeptides at increased or reduced levels as compared to non-transgenic animals of the same type (e.g., control animals). Such animals represent model systems for the study of disorders that are caused by or exacerbated by overexpression or underexpression of P3H polypeptides and for the development of therapeutic agents that modulate the expression or activity of P3H. For example, dominant-negative and constitutively activated alleles could be expressed in mice to establish physiological function.

Transgenic animals can be, for example, farm animals (pigs, goats, sheep, cows, horses, rabbits, chickens and the like) rodents (such as rats, guinea pigs, and mice), non-human primates (for example, baboons, monkeys, and chimpanzees), and domestic animals (for example, dogs and cats).

Any technique known in the art can be used to introduce a P3H transgene into animals to produce the founder lines of transgenic animals. Such techniques include, but are not limited to, pronuclear microinjection (U.S. Pat. No. 4,873,191); retrovirus mediated gene transfer into germ lines (Van der Putten et al., Proc. Natl. Acad. Sci., USA 82:6148, 1985); gene targeting into embryonic stem cells (Thompson et al., Cell 56:313, 1989); and electroporation of embryos (Lo, Mol. Cell. Biol. 3:1803, 1983). Especially useful are the methods described in Yang et al. (Proc. Natl Acac. Sci. USA 94:3004-3009, 1997).

The present invention provides transgenic animals that carry the P3H transgene in all their cells, as well as animals that carry the transgene in some, but not all of their cells. That is, the invention provides for mosaic animals. The transgene can be integrated as a single transgene or in concatamers, e.g., head-to-head tandems or head-to-tail tandems. The transgene can also be selectively introduced into and activated in a particular cell type (Lasko et al., Proc. Natl. Acad. Sci. USA 89:6232, 1992). The regulatory sequences required for such a cell-type specific activation will depend upon the particular cell type of interest, and will be apparent to those of skill in the art.

Gene targeting is useful when it is desired that a P3H transgene be integrated into the chromosomal site of an endogenous P3H gene: Briefly, when such a technique is to be used, vectors containing some nucleotide sequences homologous to an endogenous P3H gene are designed for the purpose of integrating, via homologous recombination with chromosomal sequences, into and disrupting the function of the nucleotide sequence of the endogenous gene. The transgene also can be selectively introduced into a particular cell type, thus inactivating the endogenous P3H gene in only that cell type (Gu et al., Science 265:103, 1984). The regulatory sequences required for such a cell-type specific inactivation will depend upon the particular cell type of interest, and will be apparent to those of skill in the art. These techniques are useful for preparing “knock outs” having a non-functional P3H gene.

Once transgenic animals have been generated, the expression of the recombinant P3H gene can be assayed utilizing standard techniques. Initial screening may be accomplished by Southern blot analysis or PCR techniques to determine whether integration of the transgene has taken place. The level of mRNA expression of the transgene in the tissues of the transgenic 30 animals may also be assessed using techniques which include, but are not limited to, Northern blot analysis of tissue samples obtained from the animal, in situ hybridization analysis, and RT-PCR. Samples of P3H gene-expressing tissue can also be evaluated immunocytochemically using antibodies specific for the P3H transgene product.

For a review of techniques that can be used to generate and assess transgenic animals, skilled artisans can consult Gordon (Intl. Rev. Cytol. 115:171-229, 1989), and may obtain additional guidance from, for example: Hogan et al. Manipulating the Mouse Embryo, Cold Spring Harbor Press, Cold Spring Harbor, N.Y., 1986); Krimpenfort et al. (Bio/Technology 9:86, 1991), Palmiter et al. (Cell 41:343, 1985), Kraemer et al. (Genetic Manipulation of the Early Mammalian Embryo, Cold Spring Harbor Press, Cold Spring Harbor, N.Y., 1985), Hammer et al.(Nature 315:680, 1985), Purcel et al. (Science, 244:1281, 1986), Wagner et al. (U.S. Pat. No. 5,175,385), and Krimpenfort et al. (U.S. Pat. No. 5,175,384).

### EXAMPLES

The invention is illustrated in part by the following example, which is not to be taken as limiting the invention in any way.

### Example 1

### Enzyme Characterization and Identification of Prolyl 3-Hydroxylase

In this study, prolyl 3-hydroxylase was purified from chick embryos and characterized. Two homologous gene sequences were also identified and predicted to be other members of the P3H family. The enzyme was shown to have prolyl 3-hydroxylase activity in an assay using full length procollagen. Gelatin sepharose affinity chromatography, used previously to identify proteins that bind to denatured collagen (Saga et al. J Cell Biol, 105, 517-527 (1987); Zeng et al. Biochem J, 330 (Pt 1), 109-114. (1998)), was used to demonstrate the ability of P3H1 to specifically bind to denatured collagen as well as to interact with other rER proteins as a complex. Finally, immunohistochemistry using a monoclonal antibody to P3H1 demonstrated its presence in tissues that express fibrillar collagens.

Materials and Methods

Gelatin Sepharose Affinity Chromatography and Enzyme Purification

P3H and P4H were isolated from 15 day-old chick embryos by affinity chromatography on gelatin sepharose (Pharmacia) (Saga et al., 1987; Zeng et al., 1998) with the following modifications: 12 dozen chicken embryos were mixed with an equal volume of 10 mM Tris-HCl buffer, pH 7.5, containing 0.25 M sucrose and proteinase inhibitors (5 mM EDTA, 2 mM PMSF, 2 mM N-ethylmaleimide, 1 μg/ml pepstatin A and 1 μg/ml leupeptin). Homogenization was carried out in a Waring blender at maximum speed for 3 minutes. This and all subsequent steps were performed at 4° C. The homogenate was centrifuged at 3000×g for 15 minutes in a H-6000A rotor (Sorvall). The supernatant was then centrifuged at 125,000×g for 1 hour in a 45 Ti rotor (Beckman). Resulting pellets were resuspended in twice the volume of 50 mM Tris-HOAc buffer, pH 7.5, containing 0.1% Tween 20, 0.15 M NaCl and the same protease inhibitors as described above, and treated with 1 μl/ml diisopropyl fluorophosphate (2 mM) and gently stirred overnight on ice. The extract was centrifuged at 125,000×g for 1 hour, filtered through cheesecloth and miracloth, and run over a gelatin-Sepharose 4B column (2.6×30 cm; Pharmacia) equilibrated in buffer A [50 mM Tris-HOAc buffer, pH 7.5, containing 0.2 M NaCl and 0.05 (v/v) % Tween 20]. The column was washed with at least two bed volumes of buffer A and then with two bed volumes of 50 mM Tris-HOAc buffer, pH 7.5, containing 1M NaCl and 0.05% Tween 20, followed by another bed volume of buffer A. Elution was performed using a pH gradient from 7.5 to 5.0 with buffer A. Peak fractions containing P3H and P4H were pooled, dialyzed into PBS (Life Technologies) at 4° C. and filtered through a 0.45 μm filter prior to loading onto the monoclonal antibody affinity column. Sequencing and identification of the majority of the proteins in the low pH elution peak from the gelatin sepharose affinity chromatography have been described previously (Zeng et al., 1998).

Gene Sequencing and Alignments

For the P3H1 sequencing, SDS-PAGE gels were run with the gelatin sepharose eluted material, transferred to pvdf, and stained with Coomassie blue. Protein bands of interest were cut out and either directly sequenced or proteolytically digested and resulting fragments were then sequenced. Degenerate primers were synthesized and PCR was performed using cDNA from cultured chick tendon fibroblasts from 15-day embryonic chicks (Total RNA was isolated from cells using TRIzo1 (Life Technologies). RNA was then reverse transcribed using SuperScript II Reverse Transcriptase (Invitrogen)). Resulting PCR fragments were sequenced and then used to create new primers. RACE PCR was used to clone the remainder of the gene using the Marathon cDNA amplification kit (BD Biosciences) following instructions in the user manual. Full-length sequences were verified by aligning with sequences obtained from the BBSRC chick database (see the World Wide Web at address chick.umist.ac.uk) and by repeated PCR amplification and sequencing of the P3H1 gene. Alignments were done using the Vector NTI version 7 software (InforMax, Inc).

Antibodies and Immunoaffinity Chromatography

The mouse monoclonal antibody 1C10 was generated using the pooled peak fractions from the gelatin sepharose low pH eluted material as the immunogen. The antibody was produced and selected by standard methods and specificity of the antibody was determined using immunoblotting and ELISA techniques. The antibody recognizes both nonreduced and reduced (at much lower affinity) P3H1 and was used to create an affinity column for the purpose of protein purification. Briefly, a 2 ml column was created using approximately 10 mg of the 1C10 antibody and AminoLink® plus coupling gel (Pierce) following the manufacturer's instructions. Pooled and dialyzed peak fractions from the gelatin sepharose elution were then loaded onto the antibody column and the flow through was collected and concentrated for further purification of prolyl 4-hydroxylase and PDI by sieve chromatography on superose 12 resin (Pharmacia). The 1C10 antibody column was washed with 5 volumes of PBS (procedure described in Current Protocols in Protein Science Online), and then washed with 5 volumes of wash B buffer containing 50 mM sodium phosphate, pH 6.0, 0.5 M NaCl, and 0.1% Triton X-100 to elute associated proteins. P3H1 was eluted in 50 mM glycine-HCl, pH 2.5, 150 mM NaCl, and 0.1% Triton-X 100 and then dialyzed extensively into 50 mM Tris-HCl buffer containing 0.2 M NaCl, aliquoted and frozen at −20° C. for future use in enzyme assays (see below). Sequences of proteins eluted with the wash B buffer were determined by Edman degradation in a protein sequencer (Applied Biosystems Procise Sequencer). In the case of proteins whose N-termini were blocked, peptides for sequencing were prepared by digestion with trypsin, followed by separation on a Vydac C₁₈ reversed-phase column.

Labeled Substrate Preparation and Enzyme Assays

Prolyl 3-hydroxylase activity was measured based on the amount of tritiated water (THO) formed from a labeled procollagen substrate (Kivirikko et al. Matrix Biol, 16, 357-368 (1998); Risteli et al. Anal Biochem, 84, 423-431 (1978)) with the following modifications. [2,3-³H]-L-Proline, 42 Ci/mmol, was purchased from Sigma.[2,3-³H]-proline-labeled nonhydroxylated procollagen was prepared from the isolated cells of the leg tendons from 12 dozen 15 day-old chicken embryos (Berg et al., Biochemistry, 12, 3395-3401 (1973); Dehm et al. Biochim Biophys Acta, 264, 375-382 (1972); Kivirikko et al., Methods Enzymol, 82 Pt A, 245-304 (1982)). The cells were preincubated for 30 minutes in 0.3 mM α,α′-dipyridyl and then for an additional 4 hours with 500 μCi of [2,3-³H]-L-proline. The nonhydroxylated procollagen was extracted with 0.1 M acetic acid (Berg et al., 1973). After centrifugation at 20,000×g for 30 minutes the supernatant was dialyzed at 4° C. into 50 mM Tris-HCl buffer, pH 7.8, containing 200 mM NaCl, heated to 100° C. for 10 minutes, and centrifuged at 1000×g for 10 minutes to remove the precipitate formed during heating.

The supernatant was incubated with approximately 5 μg of purified chick prolyl 4-hydroxylase for 4 hours at 37° C. in a final volume of 10 ml, containing 0.08 mM FeSO⁴, 2 mM ascorbic acid, 0.5 mM 2-oxoglutarate, and 0.05 M Tris-HCl buffer, pH 7.8, to convert all appropriate prolyl residues to 4-hydroxylprolyl residues (Tryggvason et al., Biochem Biophys Res Commun, 76, 275-281 (1976)). The solution was dialyzed into 200 mM NaCl and 50 mM Tris-HCl, pH 7.8 and then stored in aliquots at −70° C. The substrate was heated at 100° C. for 10 minutes immediately before use.

The prolyl 3-hydroxylase reactions were performed as described previously (Kivirikko et al., 1982; Risteli et al., 1978; Tryggvason et al., 1976) but with the following modifications: the enzyme reaction was carried out for 60 minutes at 24° C. in a final volume of 2.0 mls containing 1×10⁶ dpm [2,3-³H]-L-proline-labeled substrate, 0.08 mM FeSO⁴, 2 mM ascorbic acid, 0.5 mM 2-oxoglutarate, 0.2 mg/ml catalase, 2 mg/ml bovine serum albumin, 0.1 mM dithiothreitol, and 0.05 M Tris-HCl, pH 7.8. The reaction was stopped by adding 0.5 ml of 10% trichloroacetic acid and the tritiated water formed was assayed by vacuum distillation of the whole reaction mixture (Kivirikko et al., 1982; Risteli et al., 1978). A 1.8 ml aliquot of the tritiated water was mixed with 10 mls Ecolume liquid scintillation cocktail (ICN) and counted in a Beckman LS5000TD liquid scintillation counter.

The prolyl 4-hydroxylase activity was assayed by a method based on the hydroxylation-coupled decarboxylation of 2-oxo[1-¹⁴C]glutarate (Kivirikko et al., 1982). The reaction was performed in a final volume of 1.0 ml, which contained 0.1 mg (Pro-Pro-Gly)₁₀.9H²O as substrate, 2 mM ascorbic acid, 0.05 mM FeSO⁴, 0.1 mM dithiothreitol, 2 mg/ml bovine serum albumin, 0.1 mg/ml catalase, 50 mM Tris-HCl, pH 7.8, and 0.1 mM 2-oxo[1-¹⁴C]glutarate (100,000 dpm). The reaction was stopped by the addition of 1 ml of 1 M KH₂PO₄, pH 5.0.

Immunohistochemistry

Light microscopic immunohistochemical procedures were performed as has been described previously (Sakai et al., Methods Enzymol, 245, 29-52 (1994). Briefly, tissues were frozen in hexanes prior to cryosectioning. Fluoresceine isothiocyanateconjugated rabbit anti-mouse IgG (Sigma) was used for immunofluorescence microscopy, using 8-μm cryosections. The monoclonal antibody 201 against fibrillin-1 has been characterized as described previously (Reinhardt et al, J Mol Biol, 258, 104-116 (1996); Sakai et al., J Cell Biol, 103, 2499-2509 (1986); Sakai et al. Biol Chem, 266, 14763-14770 (1991).

Results

Prolyl 3-Hydroxylases are a Family of Proteins

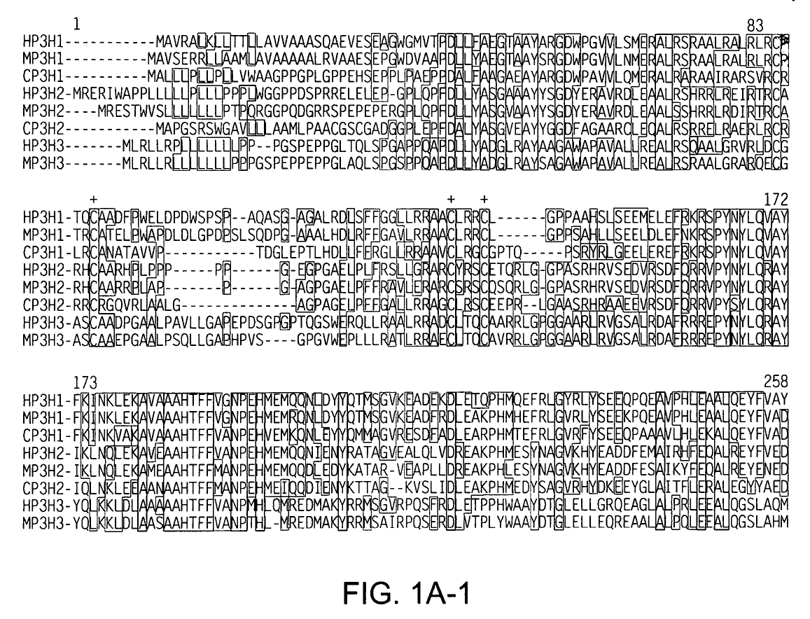

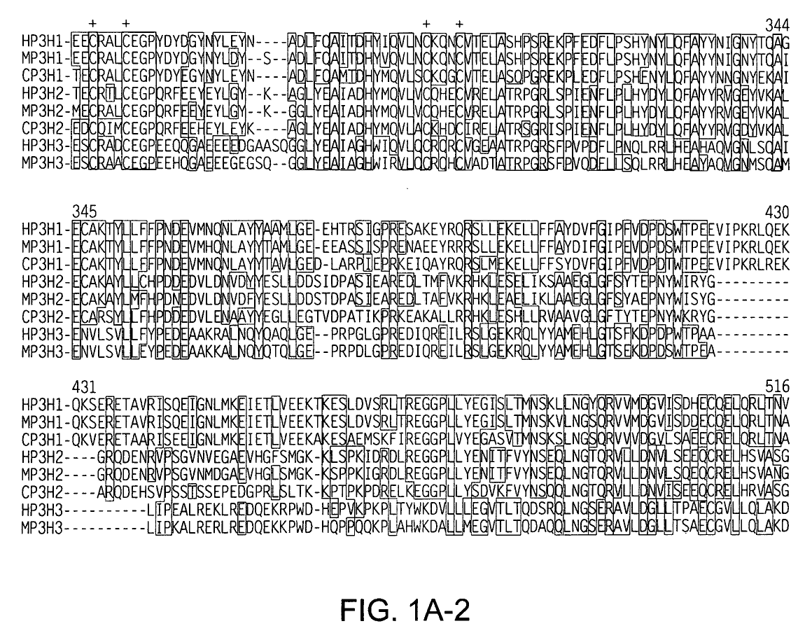

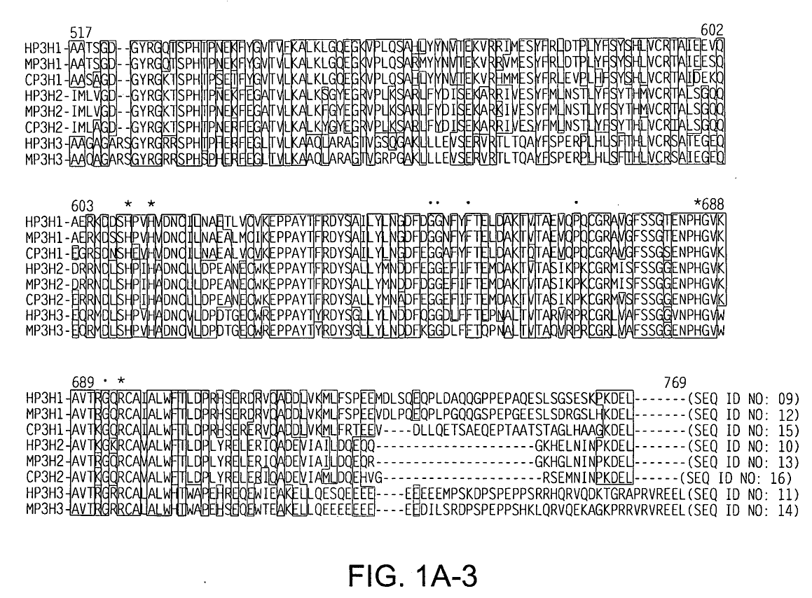

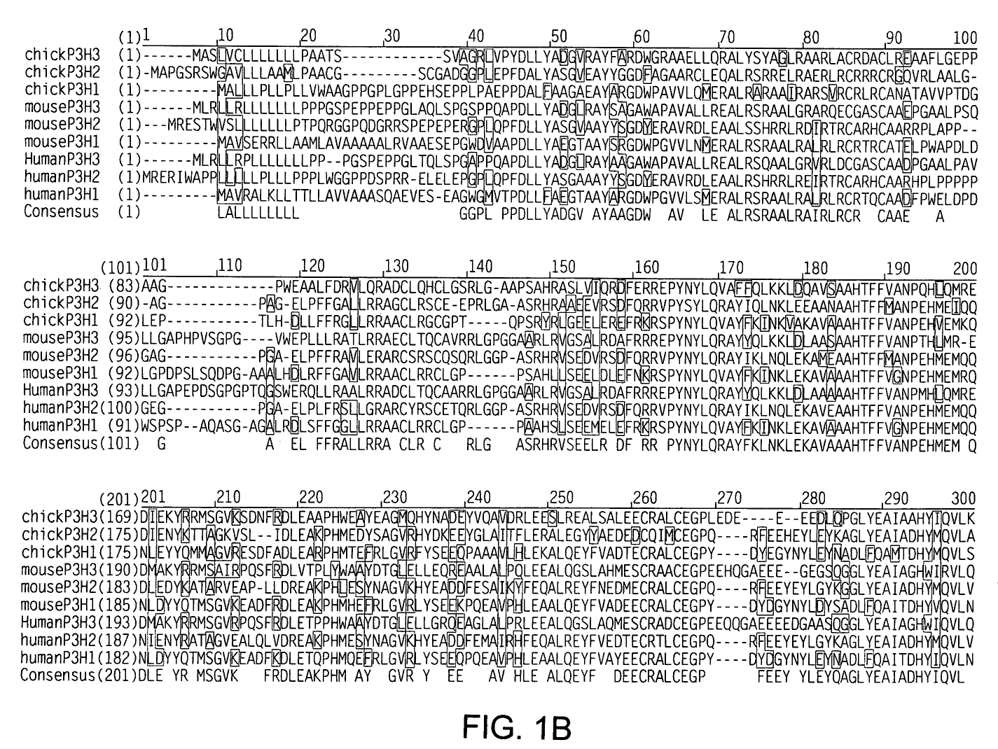

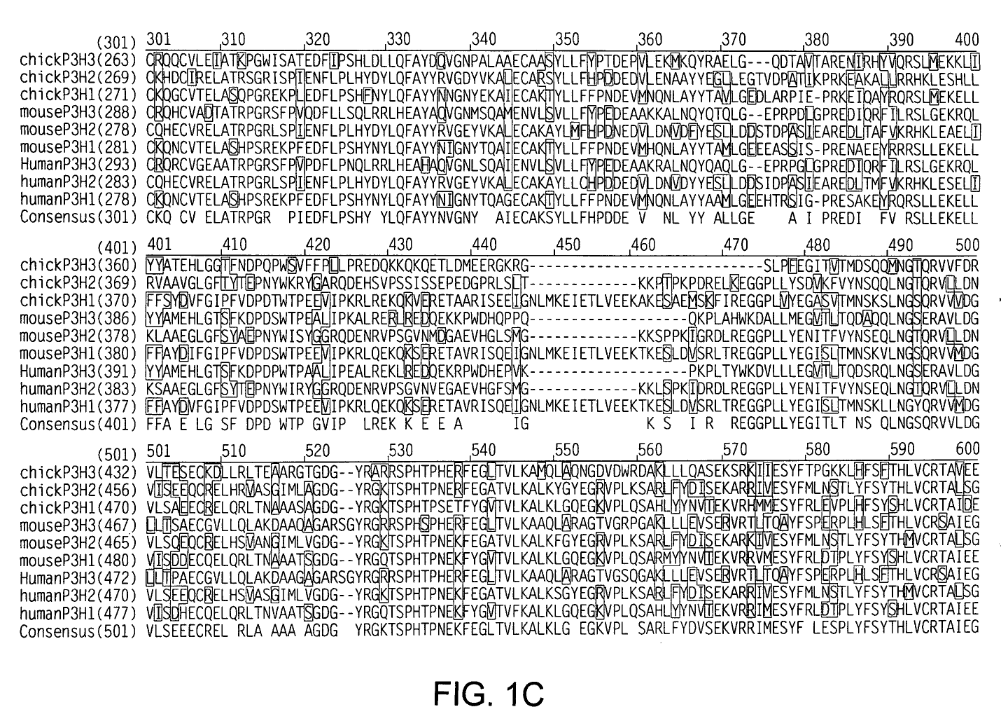

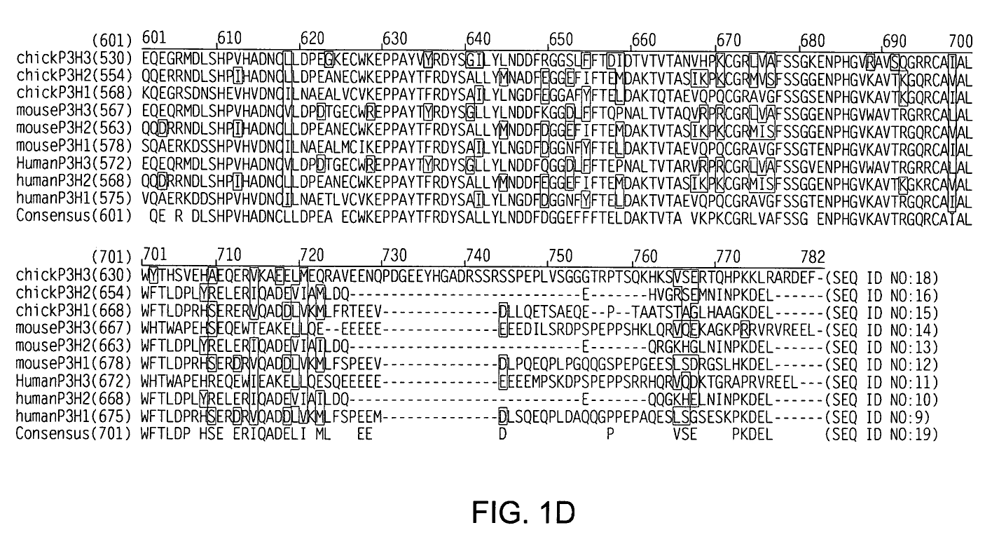

In this study, prolyl 3-hydroxylase 1 was identified as a novel rER protein present in chick embryo rER extracts partially purified by affinity chromatography on gelatin sepharose. As previously reported, proteins from rER enriched extracts can be selected according to their interactions with gelatin (denatured collagen) (Zeng et al., Biochem J. 330:109-114 (1998)). Eluted proteins were run on SDS-PAGE, transferred to PVDF membranes and bands were cut out for amino terminal sequencing. Partially purified proteins were also subjected to limited trypsin digestions to obtain internal amino acid sequences (data not shown). Degenerate primers were synthesized and PCR experiments were performed to obtain gene fragments. After an initial PCR fragment was cloned and sequenced, RACE PCR was used to clone the remainder of the gene. Sequence searches against the human and mouse genomes identified three separate genes as potential orthologs of the cloned chick protein. Further analysis of the all of the nucleotide and translated sequences demonstrated the presence of three closely-related genes in all three species (human, mouse, and chicken), one of which clearly matched the published sequence of leprecan or Gros1 and the gene cloned from chicken embryos. P3H1, 2, and 3 correspond to the human genes leprecan on chromosome 1, MLAT4 on chromosome 3, and GRCB on chromosome 12, respectively. They are classified as such based on identity, for example, the translated human sequences for P3H1 and P3H2 are 46% identical, for P3H1 and P3H3 are 41% identical, and for P3H2 and P3H3 are 38% identical. FIG. 1A is an alignment of the translated amino acid sequence of all three genes from human and mouse, and two genes from chicken. The carboxy-terminal portion of all of the molecules is highly conserved and contains critical catalytic residues shared with the lysyl and prolyl 4-hydroxylase enzymes (indicated with a “*” in FIG 1A). Other conserved residues that are shared across the P4H and LH families are indicated with a “·” in the figure. More variations are found in the amino-terminal portion of the molecules across families and species, however all family members contain four repeats of CXXXC (SEQ ID NO:26), of unknown function, in that region of the molecule (indicated by a “+” in FIG. 1A). Finally, all proteins contain the rER retention signal at their carboxy-terminal end indicating that they are likely to be resident ER proteins. FIGS. 1B-1D provide an alignment that also includes chicken P3H3, as well as a consensus sequence derived from all family members.

P3H1] Binds to Denatured Collagen and Exists in a Complex of Proteins

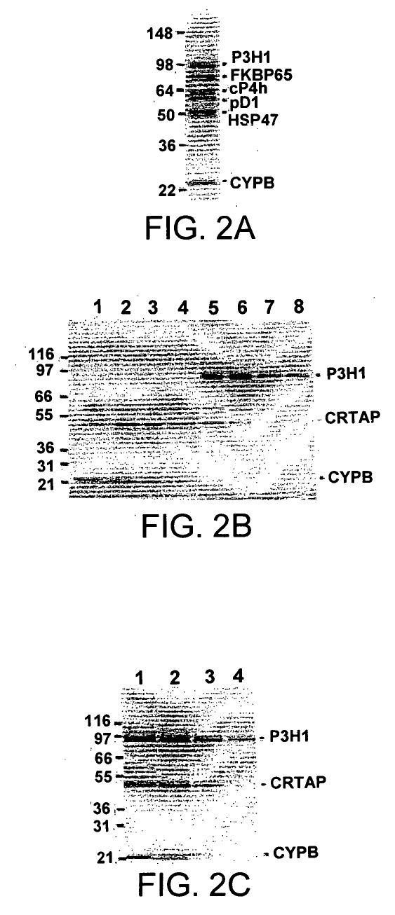

Protein extracts from the rER enriched fraction of 15 day old chick embryos bind to gelatin sepharose (Zeng et al., Biochem J, 330 (Pt 1), 109-114 (1998)). This method was initially developed as a functional assay to identify proteins or complexes of proteins that associate with unfolded or partially folded collagen in the rER during collagen biosynthesis. Molecules that specifically bound were identified and are now known to perform vital roles in the post-translational modifications and processing of the nascent procollagen molecules, such as two members of the peptidyl prolyl cis-trans isomerase family cyclophilin B (CYPB) and FKBP65, as well as HSP47 and the collagen P4H (cP4H), and PDI. After a high salt wash to remove loosely bound proteins, proteins bound to gelatin sepharose were eluted with low pH buffer (FIG. 2A).

In addition to the proteins mentioned above, another protein with an apparent molecular weight of 90 kDa on SDS-PAGE was present in the low-pH eluted material. The 90 kDa protein was cloned and sequenced and identified as the chicken homologue of leprecan or what is herein called chicken P3H1. The fact that P3H1 specifically eluted from gelatin sepharose columns suggested that it may directly bind to denatured collagen or that it exists in a complex of proteins that bind to denatured collagen.

Monoclonal antibodies were raised against the chicken P3H1 protein and it was used to make an affinity column to further purify the enzyme. During these procedures it became evident that P3H1 forms strong complexes wish other proteins eluted from the gelatin affinity column. FIG. 2B shows a reduced SDS-PAGE gel stained with Coomassie blue of the proteins that are specifically eluted off of the P3H1 antibody column. Initially, the column was loaded with the eluted extract from the gelatin sepharose affinity step, as shown in FIG. 2A, and then washed extensively in PBS. The column was then washed with a more stringent buffer (pH6 and 0.5 M NaCl) and two proteins were specifically eluted (FIG. 2B lanes 1-4). They had apparent mobilities on SDS-PAGE of 21 kDa and 46 kDa. Aminoterminal sequencing of these bands as well as tryptic digestions of the protein with a blocked amino terminus (CRTAP), identified these proteins as cyclophilin B (CYPB) and the cartilage associated protein, CRTAP. Purified P3H1 was then eluted with a low pH buffer (FIG. 2B lanes 5-8).

In FIG. 2C, the P3H1 antibody column was not washed with the mid-range buffer but eluted immediately following the PBS washes. All three proteins eluted simultaneously (CYPB, CRTAP, and P3H1), with P3H1 apparently the most abundant (FIG. 2C lanes 1-4). These results suggest not only the likelihood of P3H1 associating intracellularly with unfolded collagen molecules but also with other proteins in a specific manner.

P3H1] has Prolyl 3-hydroxylase Activity

Purified P3H1 from 15-day-old chick embryos was tested for its enzymatic activity using a labeled procollagen substrate (Kivirikko et al., 1982; Risteli et al., Eur J Biochem, 73, 485-492 (1977); Risteli et al., 1978; Tryggvason, Biochem J, 183, 303-307 (1979)). The P3H1 enzyme used in these assays was that purified without CRTAP and CYPB (as shown in FIG. 2B lanes 5-8). The only 3-hydroxyproline residues found in collagens thus far are in the sequence Gly-3(S)Hyp-4(R)Hyp-Gly- (Fietzek et al., Int Rev Connect Tissue Res, 7, 1-60 (1976); Fietzek et al., Eur J Biochem, 30, 163-168 (1972); Gaill et al., J Mol Biol, 246, 284-294 (1995); Gryder et al., J Biol Chem, 250, 2470-2474 (1975); Rexrodt et al., Eur J Biochem, 38, 384-395 (1973)). It has been reported that 3-hydroxyproline formation is dependent on the presence of4-hydroxyproline (Risteli et al., 1977; Tryggvason et al., Biochem Biophys Res Commun, 76, 275-281 (1976)) suggesting that the main substrate sequence for 3-hydroxyproline synthesis is -Gly-Pro-4Hyp-Gly-. It was therefore necessary to incubate the procollagen substrate in a large excess of prolyl 4-hydroxylase to ensure the complete conversion of all appropriate prolyl residues to 4-hydroxylproline. In the enzymatic assay used the release of tritiated water has been correlated with the formation of 3-hydroxyproline (Risteli et al., 1978) and is used as a direct measure of enzyme activity.

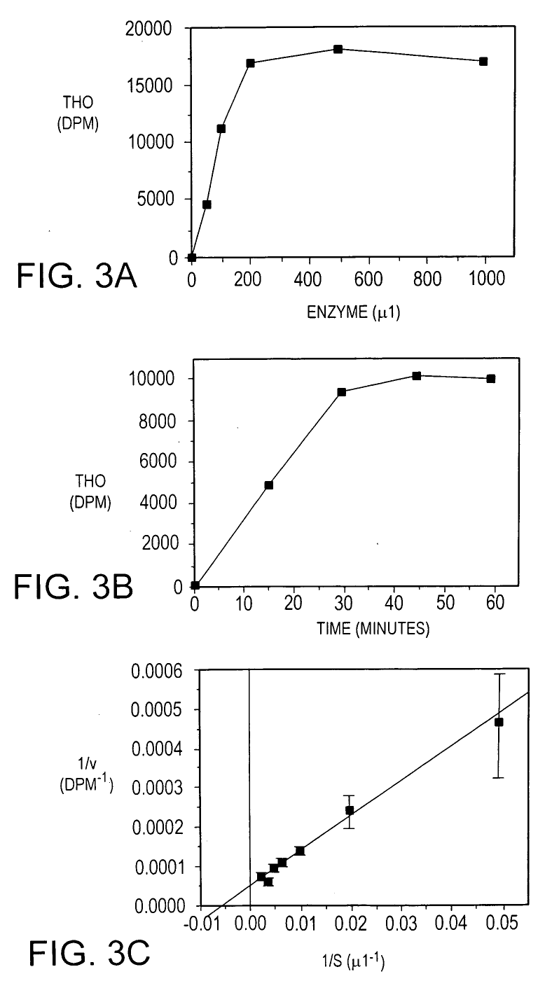

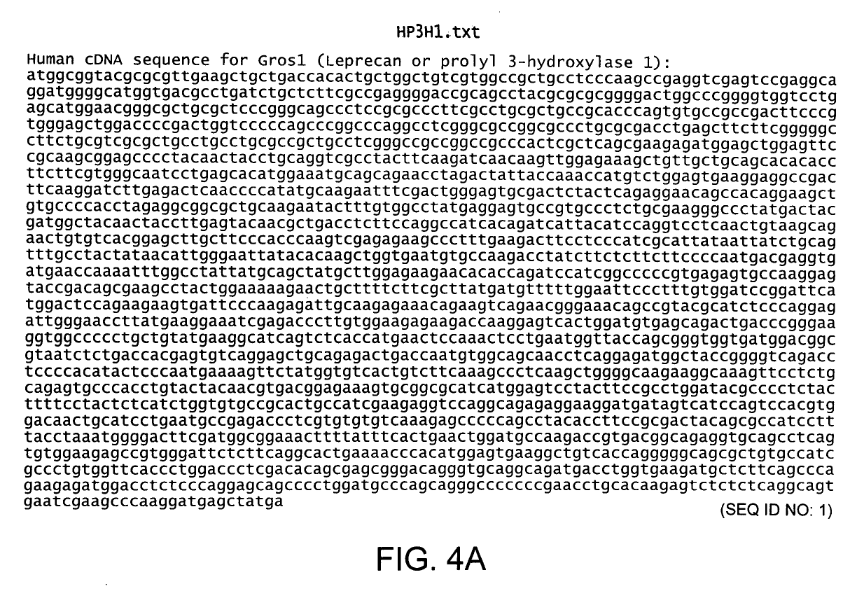

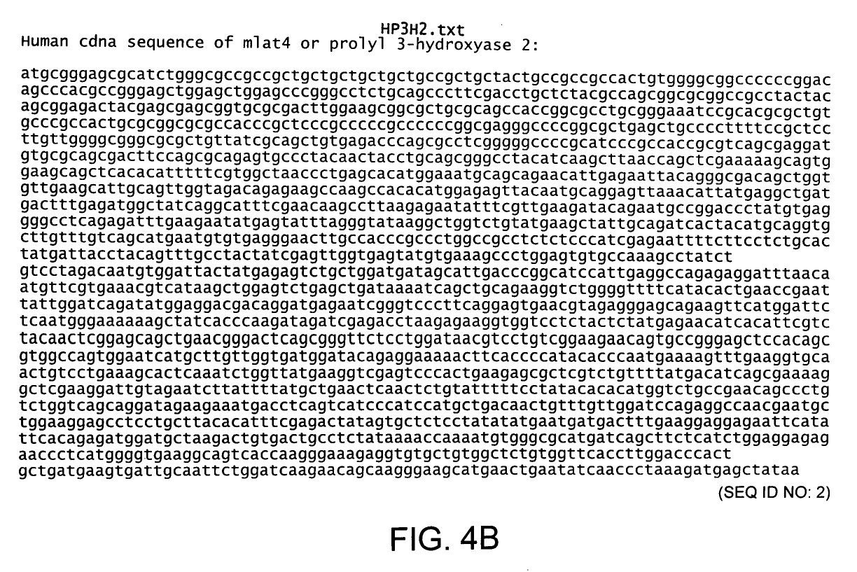

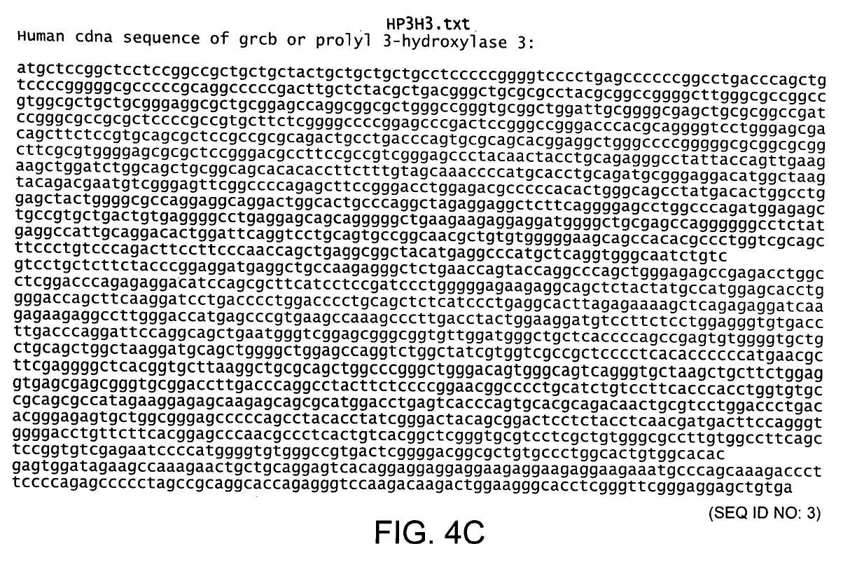

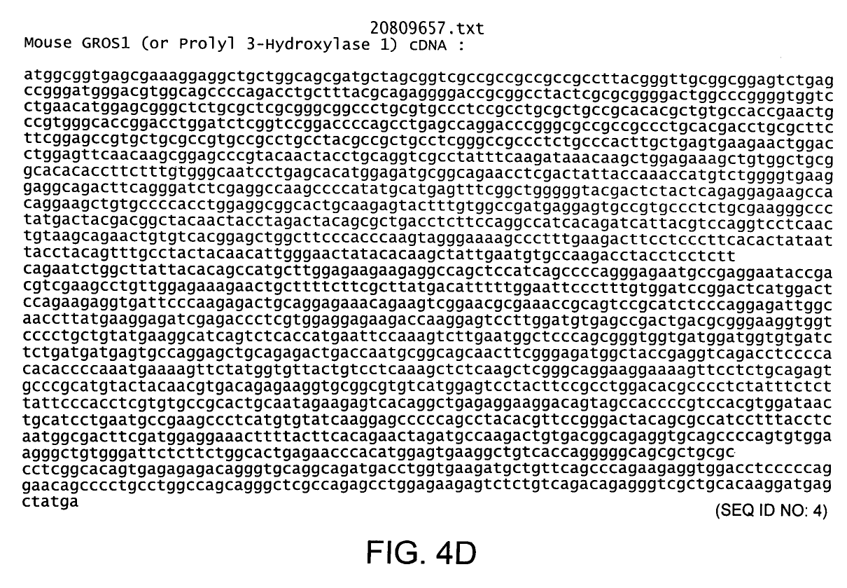

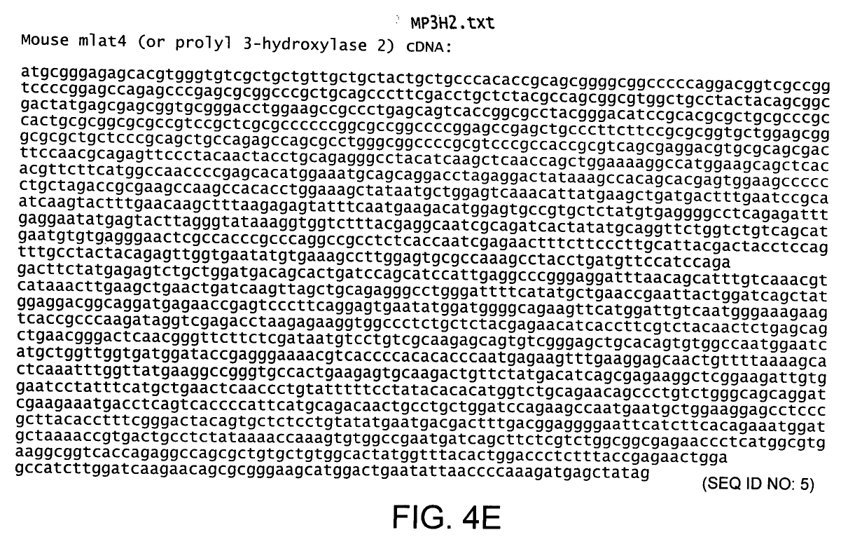

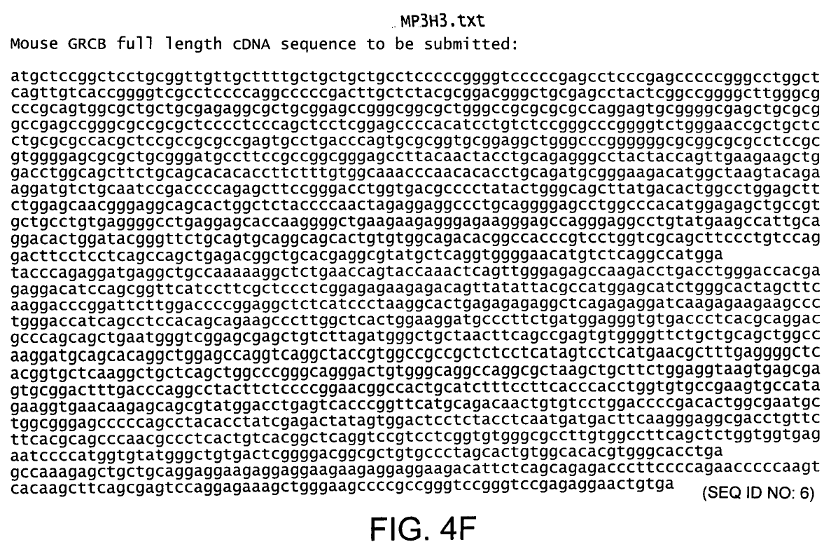

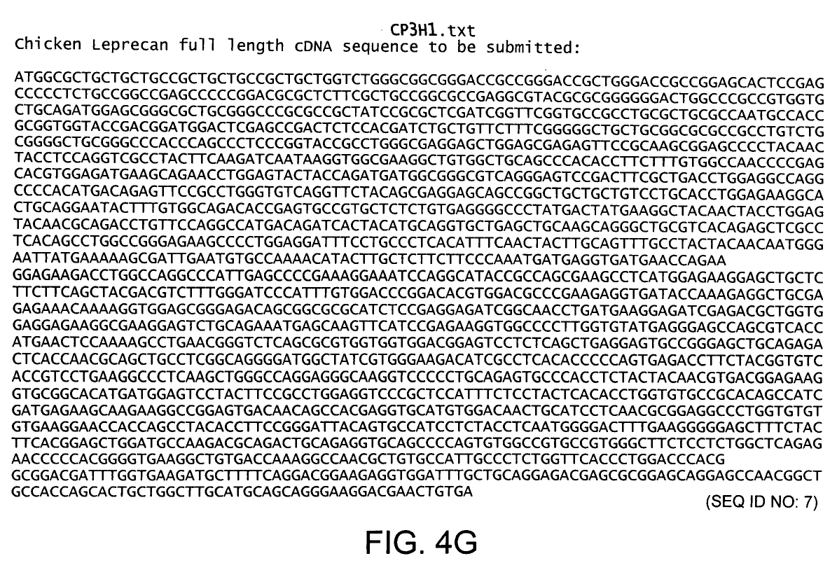

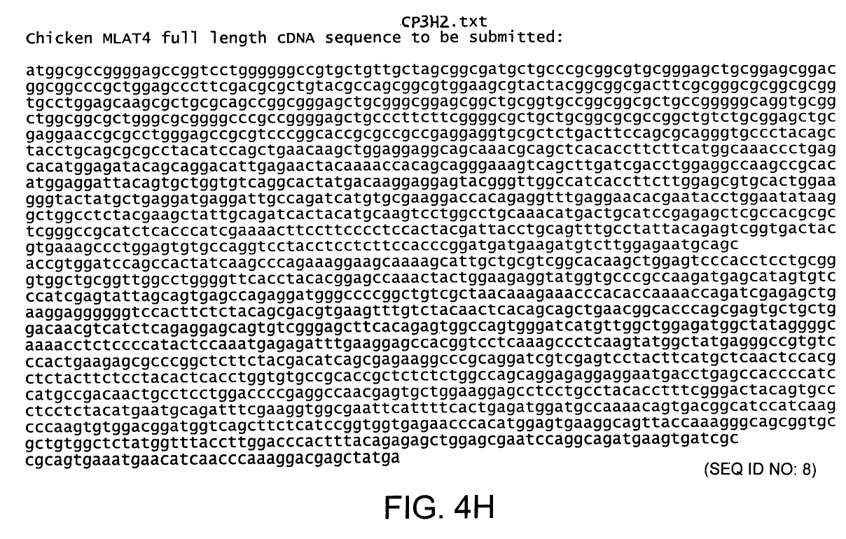

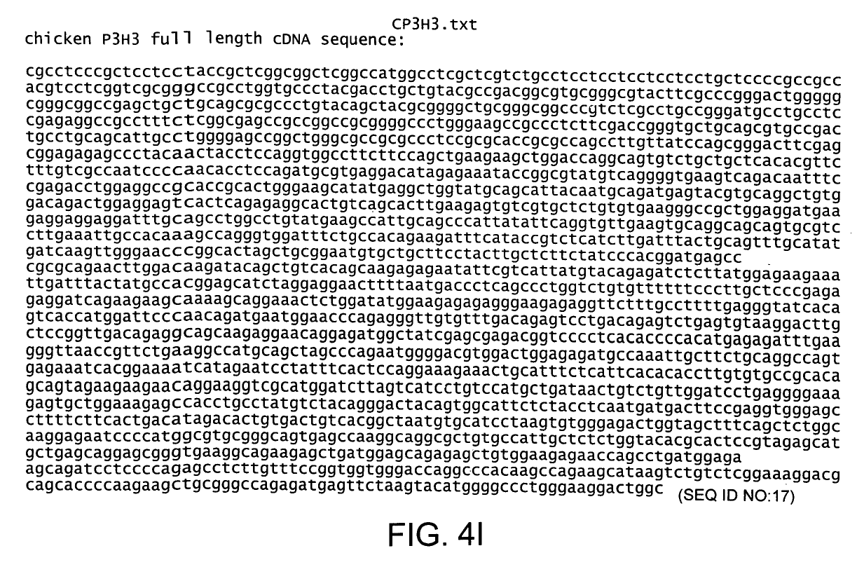

FIG. 3A demonstrates the effect of increasing enzyme concentrations (in μl of enzyme) on the formation of tritiated water (THO, measured in dpms) where enzyme activity is essentially linear with enzyme concentration up to a point where enzyme concentration is saturating (approximately 200 μl). Amino acid analysis of the purified protein determined this saturating enzyme concentration to be approximately 11.4 nM final concentration. Enzyme concentrations used in subsequent assays were performed with a concentration of enzyme where the activity is linearly related to the formation of tritiated water (75 μl of enzyme which is equal to approximately 4.3 nM final concentration of enzyme in a 2 ml reaction volume).

FIG. 3B shows the formation of tritiated water as a function of time. The reaction appears to be nearly complete by about 30 minutes. FIG. 3C shows the effect of varying substrate concentrations on the formation of tritiated water in a double reciprocal plot. Variation of the substrate concentration gave a Kₘ of 179 μl of substrate per 2 mls reaction volume or 89.5 μl of substrate per ml, which is similar to the Kₘ value previously determined for the partially purified enzyme (Risteli et al., 1978). As a control, prolyl 3-hydroxylase activity was not detected using the purified P4H enzyme in these assays indicating that there was no nonspecific release of tritiated water. Additionally, the purified P3H1 enzyme did not have any prolyl 4-hydroxylase activity when tested using the method based on the hydroxylation-coupled decarboxylation of 2-oxo[1-¹⁴C]glutarate (Kivirikko et al., 1982) excluding the possibility of it being both a P3H and a P4H.

P3H1] Localizes to Tissues that Express Fibrillar Collagens

The same monoclonal antibody used for the purification of the P3H1 enzyme was used in immunohistochemical staining of 16 day old chick embryo tissues. Embryonic chick foot was stained with the antibody 1C10 that recognizes the P3H enzyme. Clear staining for P3H1 was observed in the dermis, the tendon, and the cartilage. Additional staining with the same antibody was observed in chick cartilage. Skeletal muscle was also stained, and the distribution of P3H1 was observed to be restricted to tendon.

Embryonic chick kidney was stained with 1C10 and an antibody to fibrillin (201) as a positive control. These stainings showed restricted staining for P3H1 to the calyx but no staining for P3H1 in kidney tubules or glomeruli. Embryonic chick liver was also stained with the 1C10 and 201 (I) antibodies. Again, the presence of P3H1 appeared to be very restricted to the interlobular septum, but was largely absent from liver parenchyma. Finally, cardiac muscle was stained with 1C10 and 201, respectively. P3H1 did not appear to be present in cardiac muscle but was present in the aorta and pulmonary artery. These tissue distribution studies demonstrated the presence of P3H1 in areas where fibrillar collagens are synthesized (dermis, tendon, cartilage, large blood vessels, and connective tissue septae). In tissues like kidney cortex, liver parenchyma, and skeletal and cardiac muscle where basement membrane collagens predominate, P3H1 did not appear to be abundant, if present at all.

In Situ Studies

Expression of prolyl 3-hydroxylase 1, 2, and 3 were analyzed in the developing mouse embryo at stage E12.5 by in situ hybridizations using riboprobes made to the 3′-UTR of each gene respectively. A distinct pattern of expression was observed for the 3 genes. Prolyl 3-hydroxylase 1 localized to the precartilage/cartilaginous condensations in the vertebral bodies, as well as in Meckel's cartilage in the developing mandible, other developing facial cartilaginous structures, humerus, rib, and limb cartilage. Additionally, prolyl 3-hydroxylase 1 localized to the arch of the developing aorta.

In contrast to localization within the cartilage condensations as seen with prolyl 3-hydroxylase 1, prolyl 3-hydroxylase 2 localized to the cells residing between the vertebral bodies which will eventually differentiate to form the intervertebral discs. P3H2 was excluded from the cartilage condensations in the vertebral bodies as well as from other precartilage/cartilaginous structures throughout the embryo. P3H2 also appeared to be expressed in the smooth muscle cells underlying the epithelium in the coils of the gut and in some blood vessels, as well as in the back mesenchyme and in various parts of the developing brain.

Prolyl 3-hydroxylase 3 appeared to have a more general localization pattern and was overlapping with some areas of the other two genes. It was expressed both within the cartilage condensations of the vertebral bodies as well as in the cells surrounding them. P3H3 also seemed to be localized to the epithelial lining of the gut (in cell populations distinct from that of P3H2), as well as in the lung and kidney.

These differential expression patterns suggest unique roles for each of the prolyl 3-hydroxylase enzymes. Skilled practitioners will appreciate that a knock-out of any one of the three genes will result in an altered phenotype affecting any one or all of the tissues where expression has been observed.

The present study demonstrates that the chick homologue of leprecan (P3H1) has prolyl 3-hydroxylase activity in an assay using a labeled procollagen substrate (Risteli et al., 1978). It has been demonstrated here that P3H1 belongs to a family of proteins based on sequence alignments and high sequence homologies across three species. All three family members share conserved residues of the 2-oxoglutarate-and iron-dependent dioxygenases.

In the present example, P3H1 enzyme was partially purified using gelatin affinity chromatography. Using this method, specifically-interacting proteins were identified by amino terminal sequencing and these molecules are now known to be involved in the posttranslational modification and processing of procollagen, for example CYPB, FKBP65, HSP47, cP4H and PDI. An additional protein that specifically interacts with gelatin (denatured collagen), P3H1, was also described in this example. After being eluted at low pH from the gelatin affinity resin, P3H1 was further purified by affinity chromatography using a monoclonal antibody that specifically recognizes the P3H1. Interestingly, while purifying the enzyme in the second step other proteins were found to specifically interact with P3H1 on the antibody column, namely CYPB and CRTAP. When the column was eluted with a buffer of pH6 CYPB and CRTAP were eluted, whereas P3H1 eluted in the pH 2.5 buffer. These results suggest that P3H1 forms a tight complex with CYPB and CRTAP, however their presence is not required for full prolyl 3-hydroxylase activity, since the enzyme assays were performed in their absence and no additional enzyme activity was observed when the assays were performed in the presence of CYPB and CRTAP (data not shown). P3H1, CYPB and CRTAP, and possibly other larger complexes, may interact with unfolded procollagen chains in vivo in order to achieve a fully folded and assembled collagen molecule inside the cell.

In the present example, the amount of prolyl 3-hydroxylase activity was measured as a function of the release of tritiated water. Enzyme activity was linearly proportional to the amount of enzyme added at low to moderate enzyme concentrations (approximately up to 11.4 nM final concentration) and in the early time points (up to 30 minutes). Enzyme activity was also measured at varying substrate concentrations. When the data was plotted on a linear double-reciprocal plot the Kₘ value for this enzyme was determined to be 179 μl of substrate per 2 mls reaction volume, or 89.5 μl of substrate per ml.

Immunofluorescence was performed on day 16 chick embryos using the monoclonal antibody to P3H1. The results showed localization of the enzyme in tissues that express fibrillar collagens, for example, in tendon, cartilage, skin, and large blood vessels, but not in skeletal and cardiac muscle, kidney cortex or liver parenchyma.

Based on the results presented here, prolyl 3-hydroxylation of collagens may be due to the activity of three distinct gene products. It has been shown here that P3H1 can be purified from the rER of embryonic chick cells and is present in a complex of proteins that specifically bind to denatured collagen. Because denatured fibrillar collagen was used as the affinity substrate, and because the P3H1 immunolocalization correlates with the presence of fibrillar collagens, P3H1 likely serves to modify fibrillar collagens. It is interesting to note that unlike P4H, which requires the presence of PDI for its enzymatic activity, the presence of other interacting proteins does not appear to be necessary for P3H enzyme activity. Results presented here support the idea that P3H plays an important biological role in the folding and assembly of triple helical collagen.

### Example 2

### Comparison of Procollagen Biosynthesis in Cells Where KD90s is Suppressed by RNAi

The term RNA interference (RNAi) is used herein to describe homology-dependant gene silencing events triggered by double stranded RNA molecules. The biochemistry by which small double-stranded RNA molecules (siRNA) function has progressed significantly (Denli et al., TIBS 28:196-201 (2003)). The targeted region is selected from the given cDNA sequence beginning 50 to 100 nucleotides (nt) downstream of the start codon. For the design of the siRNA duplex a 23 nt sequence motif NAR(N17)YNN is searched for (N any nucleotide, R purine A/G, Y pyrimidine C/U) and of these motifs, sequences are selected that contain approximately 50% G/C. A range of 30 to 70% has been reported to work (Tuschl et al. The siRNA user guide (2002). Accessible on the world wide web at address mpibpc.gwdg.de). The chosen sequences are then blasted against the database to ensure that only the targeted molecule is inhibited. For each molecule, at least two independent sequences are chosen to control for the specificity of the silencing effect. Once these sequences have been identified, the molecules are synthesized or purchased (e.g., from Dharmacon Research, Lafayette, Colo.)).

For chicken KD90 and MLAT4 the following sequences are used, respectively:

CAATGCCACCGCGGTGGTACCGA (SEQ ID NO:22) (65% G/C)
and
AAGCGGAGCCCCTACAACTACCT (SEQ ID NO:23) (56%);
and
GAAGCGTACTACGGCGGCGACTT (SEQ ID NO:24) (61%)
and
GAGGAGGTGCGCTCTGACTTCCA (SEQ ID NO:25) (61%).

Transfection of siRNA duplexes is performed using OLIGOFECTAMINE® reagent (Invitrogen). For a 24-well plate, 0.84 μg of siRNA duplex is used. The siRNA duplex in MEM is mixed with OLIGOFECTAMINE reagent (3 μl in 15 μl of MEM) and incubated for 30 minutes at room temperature. The final volume is adjusted to 100 μl with MEM. This solution is then added to the cultured chick embryo cells (40 to 50% confluency). Depending on the life-time of the targeted protein, silencing will become apparent after 1 to 3 days (Tuschl et al., 2002). Silencing is tested by staining with the monoclonal antibodies and by extracting RNA and performing PCR with primers specific for the targeted gene.

Once silencing is confirmed, the cells are pulsed with [³⁵S]-methionine and [³⁵S]-cysteine for 5 minutes in folding studies or 15 minutes for secretion studies. The chase is initiated by the addition of an excess of cold methionine and cysteine. For secretion studies the chase times are selected in 10-minute increments up to one hour and 15 minute increments to 2 hours. The medium is removed at the appropriate times and analyzed by SDS PAGE electrophoresis, followed by quantitation of the radioactive bands by fluorography as described (Fessler and Fessler, 1979). For folding studies individual cell samples are lyzed and immediately treated with a mixture of trypsin and chymotrypsin for 2 minutes at 20° C. After the two minutes trypsin and chymotrypsin are inactivated by the fast addition of SDS and reducing agent and boiling for 2 minutes at 100° C. Only triple helical molecules are resistant to the proteases, and these α-chains can be analyzed by SDS PAGE and fluorography. For folding studies the chase times are 2.5, 5, 7.5, 10, 15, 20 25, 30, 45 and 60 minutes as described previously (Bächinger, 1987). Control cells treated with OLIGOFECTAMINE™ only are analyzed the same way. This work shows the rates of folding and secretion of procollagens in the presence and absence of KD90s.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. An isolated nucleic acid molecule encoding a polypeptide that:
(i) comprises at least six and less than all of the amino acids of the sequence set forth in SEQ ID NO:9, 10, 11, 12, 13, 14, 15, 16, or 18; and
(ii) displays prolyl 3-hydroxylase activity and substrate polypeptide binding ability, wherein the substrate polypeptide includes the sequence Gly-Pro-Hyp.

2. The isolated nucleic acid molecule of claim 1, wherein the substrate polypeptide includes the sequence Gly-Pro-Hyp-Gly-Ser-Gly-Ser-Gly-Lys (SEQ ID NO:20).

3. The isolated nucleic acid molecule of claim 1, wherein the substrate polypeptide is (Gly-Pro-Hyp)₄-Gly-Ser-Gly-Ser-Gly-Lys (SEQ ID NO:21).

4. The isolated nucleic acid molecule of claim 1, further comprising a nucleic acid sequence that encodes a fusion polypeptide.

5. The isolated nucleic acid molecule of claim 4, wherein the fusion partner is a hexa-histidine tag, a hemagglutinin tag, an immunoglobulin constant (Fc) region, a secretory sequence, or a detectable marker.

6. The isolated nucleic acid molecule of claim 5, wherein the detectable marker is selected from the group consisting of β-galactosidase, invertase, green fluorescent protein, luciferase, chloramphenicol, acetyltransferase, beta-glucuronidase, exo-glucanase, and glucoamylase.

7. An isolated polypeptide that (i) comprises at least six and less than all of the amino acids of the sequence set forth in SEQ ID NO:9, 10, 11, 12, 13, 14, 15, 16, or 18; and
(ii) displays prolyl 3-hydroxylase activity and substrate polypeptide binding ability, wherein the substrate polypeptide includes the sequence Gly-Pro-Hyp.

8. The polypeptide of claim 7, wherein the substrate polypeptide includes the sequence Gly-Pro-Hyp-Gly-Ser-Gly-Ser-Gly-Lys (SEQ ID NO:20).

9. The polypeptide of claim 7, wherein the substrate polypeptide is (Gly-Pro-Hyp)₄-Gly-Ser-Gly-Ser-Gly-Lys (SEQ ID NO:21).

10. The polypeptide of claim 7, further comprising a fusion polypeptide.

11. The polypeptide of claim 10, wherein the fusion polypeptide is a hexa-histidine tag, a hemagglutinin tag, an immunoglobulin constant (Fc) region, a secretory sequence, or a detectable marker.

12. The polypeptide of claim 11, wherein the detectable marker is selected from the group consisting of β-galactosidase, invertase, green fluorescent protein, luciferase, chloramphenicol, acetyltransferase, beta-glucuronidase, exo-glucanase, and glucoamylase.

13. A fusion protein comprising:
(i) a first amino acid sequence comprising a prolyl 3 hydroxylase polypeptide or fragment thereof; and
(ii) a second amino acid sequence unrelated to the first amino acid sequence, wherein the fusion protein displays prolyl 3-hydroxylase activity and substrate polypeptide binding ability, wherein the substrate polypeptide includes the amino acid sequence Gly-Pro-Hyp.

14. The fusion protein of claim 13, wherein the first amino acid sequence comprises SEQ ID NO:9, 10, 11, 12, 13, 14, 15, 16, or 18, or a fragment thereof.

15. The fusion protein of claim 13, wherein the substrate protein includes the amino acid sequence Gly-Pro-Hyp-Gly-Ser-Gly-Ser-Gly-Lys (SEQ ID NO:20).

16. The fusion protein of claim 13, wherein the substrate protein includes the amino acid sequence (Gly-Pro-Hyp)₄-Gly-Ser-Gly-Ser-Gly-Lys (SEQ ID NO:20).

17. The fusion protein of claim 13, wherein the substrate protein is procollagen or fragment thereof.

18. The fusion protein of claim 13, wherein the second amino acid sequence is a hexa-histidine tag, a hemagglutinin tag, an immunoglobulin constant (Fc) region, a secretory sequence, or a detectable marker.

19. An isolated nucleic acid sequence that encodes a polypeptide comprising SEQ ID NO:15 SEQ ID NO:16, or SEQ ID NO: 18, or a substrate binding domain- or catalytic domain-encoding fragment of SEQ ID NO:15 SEQ ID NO:16, or SEQ ID NO:18.

20. The isolated nucleic acid sequence of claim 19, wherein the sequence comprises SEQ ID NO:7, SEQ ID NO:8, or SEQ ID NO:17, or a substrate binding domain- or catalytic domain-encoding fragment thereof.

21. An isolated polypeptide comprising SEQ ID NO:15, SEQ ID NO:16, or SEQ ID NO:18, or a biologically active fragment of SEQ ID NO:15, SEQ ID NO:16, or SEQ ID NO:18.

22. A method for identifying a candidate compound that modulates prolyl 3-hydroxylase activity, the method comprising:
(a) providing a polypeptide that:
(i) comprises a prolyl 3-hydrroxylase polypeptide or a fragment thereof; and
(ii) displays prolyl 3-hydroxylase activity and substrate polypeptide binding ability;
(b) contacting the polypeptide with the substrate protein in the presence of a test compound; and
(c) comparing the level of prolyl 3-hydroxylase activity or binding activity of the polypeptide toward the substrate polypeptide in the presence of the test compound with the level of prolyl 3-hydroxylase activity or binding activity in the absence of the test compound, wherein a different level of binding or hydroxylase activity in the presence of the test compound than in its absence indicates that the test compound is a candidate compound that modulates prolyl 3-hydroxylase activity.

23. The method of claim 22, wherein the polypeptide of (a) comprises SEQ ID NO:9, 10, 11, 12, 13, 14, 15, 16, or 18, or a biologically active fragment thereof.

24. The method of claim 22, wherein the substrate polypeptide includes the amino acid sequence Gly-Pro-Hyp

25. The method of claim 22, wherein the substrate polypeptide comprises the amino acid sequence Gly-Pro-Hyp-Gly-Ser-Gly-Ser-Gly-Lys (SEQ ID NO:20).

26. The method of claim 22, wherein the substrate polypeptide includes the amino acid sequence (Gly-Pro-Hyp)₄-Gly-Ser-Gly-Ser-Gly-Lys (SEQ ID NO:21).

27. The method of claim 22, further comprising:
(d) determining whether the candidate compound modulates in vivo the activity of a prolyl 3-hydroxylase polypeptide or collagen biosynthesis, wherein modulation indicates that the candidate compound is a prolyl 3-hyrdoxylase modulating agent.

28. The method of claim 22, wherein the test compound is selected from the group consisting of polypeptides, ribonucleic acids, small molecules, and deoxyribonucleic acids.

29. The method of claim 19, wherein:
(a) the polypeptide is provided as a first fusion protein comprising the polypeptide fused to (i) a transcription activation domain of a transcription factor or (ii) a DNA-binding domain of a transcription factor;
(b) the substrate protein is provided as a second fusion protein comprising a substrate protein fused to (i) a transcription activation domain of a transcription factor or (ii) a DNA-binding domain of a transcription factor, to interact with the first fusion protein; and
binding of the polypeptide with the substrate polypeptide is detected as reconstitution of a transcription factor.

30. A method for identifying a candidate compound that modulates prolyl 3-hydroxylase activity, the method comprising:
(a) providing a polypeptide comprising a prolyl 3-hydroxylase protein or fragment thereof;
(b) contacting the polypeptide or fragment thereof with a test compound; and
(c) detecting binding between the polypeptide or fragment thereof with the test compound, wherein binding indicates that the test compound is a candidate compound that modulates prolyl 3-hydroxylase activity.

31. The method of claim 30, wherein the polypeptide comprises the sequence set forth in SEQ ID NO:9, 10, 11, 12, 13, 14, 15, 16, or 18, or a fragment thereof.

32. The method of claim 30, wherein the test compound is immobilized and binding of the polypeptide to the test compound is detected as immobilization of the polypeptide on the immobilized test compound.

33. The method of claim 30, further comprising:
(d) determining whether the candidate compound modulates in vivo the activity of a prolyl 3-hydroxylase polypeptide or collagen biosynthesis, wherein modulation indicates that the candidate compound is a prolyl 3-hyrdoxylase modulating agent.

34. The method of claim 30, wherein the test compound is selected from the group consisting of polypeptides, ribonucleic acids, small molecules, and deoxyribonucleic acids.

35. The method of claim 30, wherein:
(a) the polypeptide is provided as a first fusion protein comprising the polypeptide fused to (i) a transcription activation domain of a transcription factor or (ii) a DNA-binding domain of a transcription factor;
(b) the test compound is provided as a second fusion protein comprising a test protein fused to (i) a transcription activation domain of a transcription factor or (ii) a DNA-binding domain of a transcription factor, to interact with the first fusion protein; and
binding of the polypeptide with the test compound is detected as reconstitution of a transcription factor.

36. A pharmaceutical formulation comprising a candidate compound identified by the method of claim 22, and a pharmaceutically acceptable excipient.

37. A pharmaceutical formulation comprising a candidate compound identified by the method of claim 30, and a pharmaceutically acceptable excipient.

38. A method of modulating collagen biosynthesis in an organism, the method comprising administering to the organism a therapeutically effective amount of the pharmaceutical formulation of claim 36.

39. A method for modulating collagen biosynthesis in an organism, the method comprising suppressing expression of prolyl 3-hydroxylase in the organism using an siRNA molecule.

40. The method of claim 39, wherein the target of the siRNA molecule comprises the sequence:
(1) CAATGCCACCGCGGTGGTACCGA; (SEQ ID NO:22)
(2) AAGCGGAGCCCCTACAACTACCT; (SEQ ID NO:23)
(3) GAAGCGTACTACGGCGGCGACTT; (SEQ ID NO:24)
or
(4) GAGGAGGTGCGCTCTGACTTCCA. (SEQ ID NO:25)

41. An isolated antibody that specifically binds to the polypeptide of claim 7.

42. A transgenic non-human mammal, one or more of whose cells comprise a transgene encoding Prolyl 3-hydroxylase 2 (P3H2) or Prolyl 3-hyrdoxylase 3 (P3H3), wherein the transgene is expressed in one or more cells of the transgenic mammal such that the mammal exhibits a P3H2- or P3H3-mediated disorder.
